(19) United States
(12) Patent Application Publication
(10) Pub. No.: US 2026/0226724 A1
(43) Pub. Date: Aug. 6, 2026

(54) BIOLOGICAL INFORMATION MEASUREMENT SYSTEM AND TOILET SEAT DEVICE

(71) Applicant: TOTO LTD., Kitakyushu-shi, Fukuoka (JP)

(72) Inventors: Koki Nagano, Fukuoka (JP); Shota Fujino, Fukuoka (JP); Haruna Kawasaki, Fukuoka (JP); Masamichi Tosaki, Fukuoka (JP); Mitsuhiro Shimada, Fukuoka (JP)

(21) Appl. No.: 19/149,566

(22) PCT Filed: Jan. 19, 2024
(86) PCT No.: PCT/JP2024/001492

§ 371 Date: Jul. 21, 2025

(30) Foreign Application Priority Data
Mar. 24, 2023 (JP) 2023-047626

(51) Int. Cl. E03D 9/00

(52) U.S. Cl. CPC E03D 9/00

(57) ABSTRACT

A biological information measurement system according to an embodiment including: a gas detection device that includes a first gas sensor that reacts with a hydrogen gas contained in a gas mixture, and a second gas sensor that reacts with an odoriferous gas containing a sulfur component and the hydrogen gas; and a control device that controls the gas detection device, in which the control device calculates a second calculation value corresponding to the hydrogen gas of the second gas sensor based on a plurality of calculation values corresponding to the hydrogen gas contained in the gas mixture, and calculates a third calculation value corresponding to the odoriferous gas based on a detection result of the second gas sensor and the second calculation value, and the plurality of calculation values include a first calculation value obtained based on a detection result of the first gas sensor.

### FIELD

The disclosed embodiment relates to a biological information measurement system and a toilet seat device.

### BACKGROUND

Hitherto, there has been known a health measurement device that senses fecal gas emitted simultaneously with feces, measures a hydrogen sulfide gas, which is an example of an odoriferous gas contained in the fecal gas, and measures an intestinal state from the hydrogen sulfide gas (see, for example, Patent Literature 1). In addition, a biological information system that analyzes a daily physical condition of a measurer from data of a health-related gas including at least one of a hydrogen gas, a carbon dioxide gas, or a methane gas and the odoriferous gas containing a sulfur component is known, the health-related gas and the odoriferous gas being contained in the fecal gas (see, for example, Patent Literature 2). For example, there is provided a technology for calculating an amount of the odoriferous gas by separating an influence of the hydrogen gas detected by a hydrogen gas sensor from a detection result based on measurement using the odoriferous gas in order to suppress the influence of the hydrogen gas when detecting the odoriferous gas.

### CITATION LIST

### Patent Literature

Patent Literature 1: JP 2009-250922 A
Patent Literature 2: JP 6674623 B2

### SUMMARY

### Technical Problem

However, there is room for improvement in the above-described technologies according to the related art. For example, by simply separating the influence of the hydrogen gas detected by the hydrogen gas sensor from the detection result based on the measurement using the odoriferous gas, appropriate gas measurement may be difficult due to measurement variations of the hydrogen gas sensor. Therefore, it is desired to appropriately execute processing based on gas measurement.

An object of the disclosed embodiment is to provide a biological information measurement system and a toilet seat device capable of appropriately executing processing based on gas measurement.

### Solution to Problem

A biological information measurement system according to one aspect of an embodiment that measures biological information of a user in a toilet room based on fecal gas emitted into a bowl of a closet bowl installed in the toilet room, the biological information measurement system includes a gas detection device that includes a first gas sensor that reacts with a hydrogen gas contained in a gas mixture, and a second gas sensor that reacts with an odoriferous gas containing a sulfur component and the hydrogen gas; and a control device that controls the gas detection device, wherein the control device calculates a second calculation value corresponding to the hydrogen gas of the second gas sensor based on a plurality of calculation values corresponding to the hydrogen gas contained in the gas mixture, and calculates a third calculation value corresponding to the odoriferous gas based on a detection result of the second gas sensor and the second calculation value, the biological information measurement system estimates a health condition of the user or information regarding the health condition based on the third calculation value, and the plurality of calculation values include a first calculation value corresponding to the hydrogen gas obtained based on a detection result of the first gas sensor.

With the biological information measurement system according to one aspect of the embodiment, the second calculation value is acquired based on an estimated value obtained from the plurality of calculation values derived from the hydrogen gas and including the first calculation value derived from the hydrogen gas and obtained based on a detection result of the first gas sensor. Therefore, an influence of measurement variations of a hydrogen gas sensor (corresponding to the first gas sensor) can be reduced, and an amount of the hydrogen gas closer to a true value can be calculated. Therefore, with the biological information measurement system according to one aspect of the embodiment, in a configuration in which an amount of the odoriferous gas is calculated by a method of removing an influence of the hydrogen gas from a detection value of an odoriferous gas sensor based on the amount of the hydrogen gas detected by the hydrogen gas sensor, even if a detection amount of the hydrogen gas fluctuates due to the measurement variations of the hydrogen gas sensor, a detection amount of the odoriferous gas can be suppressed from becoming 0 or less due to the hydrogen gas, and for example, the health condition such as a condition of an intestinal environment can be accurately calculated. Therefore, the biological information measurement system can appropriately execute processing based on gas measurement.

As described in Patent Literature 1 and Patent Literature 2 above, the inventors have continued studies of physical condition measurement using information of the fecal gas, and in the studies, it has been found that a temporal variation in a ratio between a health-related gas including hydrogen, carbon dioxide, acetic acid, methane, ethanol, water, and the like and the odoriferous (malodorous) gas including ammonia, trimethylamine, hydrogen sulfide, methyl mercaptan, indole, skatole, and the like, which are contained in the fecal gas (flatulence) emitted during defecation, indirectly captures a temporal variation in the intestinal environment. However, in a case where the amount of the hydrogen gas detected from the fecal gas is large or the amount of the odoriferous gas is small, when an attempt is made to simply separate the influence of the hydrogen gas detected by the hydrogen gas sensor from the detection value of the odoriferous gas detected by the odoriferous gas due to measurement variations of the hydrogen gas sensor as in Patent Literature 2, the detection value of the odoriferous gas becomes 0 or a negative value if a variation of the detection amount obtained by the hydrogen gas sensor has a positive value, and thus, the intestinal environment cannot be correctly estimated, which is problematic. Therefore, with the biological information measurement system according to one aspect of the embodiment, even if the amount of the hydrogen gas fluctuates due to the measurement variations of the hydrogen gas sensor, it is possible to suppress a large error in the amount of the odoriferous gas.

In the biological information measurement system according to one aspect of the embodiment, includes a suction device that sucks the gas mixture in the bowl; and sealing means that holds a gas sucked by the suction device in a sealed space, wherein the second calculation value is a statistical value of the plurality of calculation values obtained by measuring the gas in the sealed space a plurality of times by using the first gas sensor.

With the biological information measurement system according to one aspect of the embodiment, information regarding the same gas can be acquired a plurality of times by the same sensor by holding the fecal gas from a single emission event in the sealed space using the sealing means and acquiring the information a plurality of times from the gas in the sealed space. As a result, with the biological information measurement system according to one aspect of the embodiment, the detection amount of the hydrogen gas can be accurately detected regardless of the measurement variations of the hydrogen gas sensor itself. Therefore, the biological information measurement system can appropriately execute processing based on gas measurement.

In the biological information measurement system according to one aspect of the embodiment, the sealing means includes a storage portion that stores the gas sucked by the suction device, and the second calculation value is a statistical value of the plurality of calculation values obtained by measuring the gas in the storage portion a plurality of times.

With the biological information measurement system according to one aspect of the embodiment, a sensor output can be stabilized with a simple configuration by continuously bringing the gas held in the flow path into contact with the sensor. Therefore, the biological information measurement system can appropriately execute processing based on gas measurement.

In the biological information measurement system according to one aspect of the embodiment, the sealing means has a flow path switchable to a closed flow path by opening/closing means, and the second calculation value is a statistical value of the plurality of calculation values obtained by measuring a gas in the flow path a plurality of times.

With the biological information measurement system according to one aspect of the embodiment, the gas in the flow path can be repeatedly measured by the first gas sensor, and a plurality of pieces of information can be acquired without providing the storage portion. Therefore, the biological information measurement system can appropriately execute processing based on gas measurement.

In the biological information measurement system according to one aspect of the embodiment, includes a third gas sensor that has higher sensitivity to the hydrogen gas and lower sensitivity to the odoriferous gas than the second gas sensor, wherein the plurality of calculation values include the first calculation value and a fourth calculation value corresponding to the hydrogen gas obtained based on a detection result of the third gas sensor.

With the biological information measurement system according to one aspect of the embodiment, it is possible to average pieces of information regarding the gas corresponding to the same timing in the fecal gas from a single emission event by using the pieces of information of a plurality of sensors, and it is possible to detect the detection amount of the hydrogen gas more accurately. Therefore, the biological information measurement system can appropriately execute processing based on gas measurement.

In the biological information measurement system according to one aspect of the embodiment, the third gas sensor is a gas sensor that reacts with a methane gas, and the control device uses the third gas sensor as a sensor for detecting the methane gas in a case where it is determined that the methane gas is contained in the fecal gas of the user, and uses the third gas sensor as a sensor for detecting the hydrogen gas in a case where it is determined that the methane gas is not contained in the fecal gas of the user.

With the biological information measurement system according to one aspect of the embodiment, it is possible to use a sensor that can be used for other purposes without providing a plurality of the same sensors, and thus, usability can be improved. Therefore, the biological information measurement system can appropriately execute processing based on gas measurement.

A biological information measurement system according to one aspect of an embodiment that measures biological information of a user in a toilet room based on fecal gas emitted into a bowl of a closet bowl installed in the toilet room, the biological information measurement system includes a gas detection device that includes a first gas sensor that reacts with a hydrogen gas contained in a gas mixture, and a second gas sensor that reacts with an odoriferous gas containing a sulfur component and the hydrogen gas; and a control device that controls the gas detection device, wherein the control device calculates a second calculation value corresponding to the hydrogen gas of the second gas sensor based on a plurality of calculation values corresponding to the hydrogen gas contained in the gas mixture, and calculates a third calculation value corresponding to the odoriferous gas based on a detection result of the second gas sensor and the second calculation value, the biological information measurement system estimates a health condition of the user or information regarding the health condition by using a score calculated based on the third calculation value, and the score is calculated based on a ratio between a value based on a first calculation value corresponding to the hydrogen gas obtained based on a detection result of the first gas sensor corresponding to detection at a common timing in defecation of the user, and a value based on the third calculation value.

With the biological information measurement system according to one aspect of the embodiment, the final intestinal health is calculated by acquiring and averaging a plurality of sets of health result information obtained by acquiring the hydrogen gas and the odoriferous gas at the same timing, so that the influence of the measurement variations of the hydrogen gas sensor itself can be reduced, and for example, the health condition such as the condition of the intestinal environment can be accurately calculated. Therefore, the biological information measurement system can appropriately execute processing based on gas measurement.

A toilet seat device according to one aspect of an embodiment that measures biological information of a user in a toilet room based on fecal gas emitted into a bowl of a closet bowl installed in the toilet room, the toilet seat device includes a gas detection device that includes a first gas sensor that reacts with a hydrogen gas contained in a gas mixture, and a second gas sensor that reacts with an odoriferous gas containing a sulfur component and the hydrogen gas; and a control device that controls the gas detection device, wherein the control device calculates a second calculation value corresponding to the hydrogen gas of the second gas sensor based on a plurality of calculation values corresponding to the hydrogen gas contained in the gas mixture, and calculates a third calculation value corresponding to the odoriferous gas based on a detection result of the second gas sensor and the second calculation value, the toilet seat device estimates a health condition of the user or information regarding the health condition based on the third calculation value, and the plurality of calculation values include a first calculation value corresponding to the hydrogen gas obtained based on a detection result of the first gas sensor.

With the toilet seat device according to one aspect of the embodiment, the second calculation value is acquired based on an estimated value obtained from the plurality of calculation values derived from the hydrogen gas and including the first calculation value derived from the hydrogen gas and obtained based on a detection result of the first gas sensor. Therefore, the influence of the measurement variations of the hydrogen gas sensor can be reduced, and the amount of the hydrogen gas closer to a true value can be calculated. Therefore, with the toilet seat device according to one aspect of the embodiment, in a configuration in which the amount of the odoriferous gas is calculated by a method of removing the influence of the hydrogen gas from the detection value of the odoriferous gas sensor based on the amount of the hydrogen gas detected by the hydrogen gas sensor, even if the detection amount of the hydrogen gas fluctuates due to the measurement variations of the hydrogen gas sensor, the detection amount of the odoriferous gas can be suppressed from becoming 0 or less due to the hydrogen gas, and for example, the health condition such as the condition of the intestinal environment can be accurately calculated. Therefore, the toilet seat device can appropriately execute processing based on gas measurement.

A toilet seat device according to one aspect of an embodiment that measures biological information of a user in a toilet room based on fecal gas emitted into a bowl of a closet bowl installed in the toilet room, the toilet seat device includes a gas detection device that includes a first gas sensor that reacts with a hydrogen gas contained in a gas mixture, and a second gas sensor that reacts with an odoriferous gas containing a sulfur component and the hydrogen gas; and a control device that controls the gas detection device, wherein the control device calculates a second calculation value corresponding to the hydrogen gas of the second gas sensor based on a plurality of calculation values corresponding to the hydrogen gas contained in the gas mixture, and calculates a third calculation value corresponding to the odoriferous gas based on a detection result of the second gas sensor and the second calculation value, the toilet seat device estimates a health condition of the user or information regarding the health condition by using a score calculated based on the third calculation value, and the score is calculated based on a ratio between a value based on a first calculation value corresponding to the hydrogen gas obtained based on a detection result of the first gas sensor corresponding to detection at a common timing in defecation of the user, and a value based on the third calculation value.

With the toilet seat device according to one aspect of the embodiment, the final intestinal health is calculated by acquiring and averaging a plurality of sets of health result information obtained by acquiring the hydrogen gas and the odoriferous gas at the same timing, so that the influence of the measurement variations of the hydrogen gas sensor itself can be reduced, and for example, the health condition such as the condition of the intestinal environment can be accurately calculated. Therefore, the toilet seat device can appropriately execute processing based on gas measurement.

### Advantageous Effects of Invention

According to an aspect of the embodiment, processing based on gas measurement can be appropriately executed.

### BRIEF DESCRIPTION OF DRAWINGS

FIG. 1 is a perspective view illustrating an example of a configuration of a toilet room according to an embodiment.

FIG. 2 is a plan view illustrating an example of a configuration of a measurement device according to the embodiment.

FIG. 3 is a diagram illustrating an example of an overall outline of a biological information measurement system according to the embodiment.

FIG. 4 is a diagram illustrating an example of a relationship between an action of a user and an operation of the system.

FIG. 5 is a block diagram illustrating an example of a configuration of a toilet seat device according to the embodiment.

FIG. 6 is a block diagram illustrating an example of a configuration of a control device according to the embodiment.

FIG. 7 is a diagram illustrating an example of a configuration of a gas sensor.

FIG. 8 is a diagram illustrating an example of a relationship between a value based on measurement performed by the gas sensor and an amount of a gas.

FIG. 9 is a diagram illustrating an example of the gas sensor and a reacting component.

FIG. 10 is a diagram illustrating an example of processing of calculating an amount of an odoriferous gas.

FIG. 11 is a diagram illustrating an outline of the calculation of the amount of the odoriferous gas.

FIG. 12 is a diagram illustrating an example of an influence of measurement variations of the gas sensor on the calculation.

FIG. 13 is a diagram illustrating an example of the influence of the measurement variations of the gas sensor on the calculation.

FIG. 14 is a diagram illustrating a first measurement example using the gas sensor.

FIG. 15 is a diagram illustrating a second measurement example using the gas sensor.

FIG. 16 is a diagram illustrating a third measurement example using the gas sensor.

FIG. 17 is a diagram illustrating a fourth measurement example using the gas sensor.

FIG. 18 is a diagram illustrating fifth and sixth measurement examples using the gas sensor.

FIG. 19 is a diagram illustrating a seventh measurement example using the gas sensor.

FIG. 20 is a diagram illustrating an example of a configuration and control corresponding to the third measurement example.

FIG. 21 is a diagram illustrating an example of a configuration and control corresponding to the fourth measurement example.

FIG. 22 is a diagram illustrating an example of the configuration and the control corresponding to the fourth measurement example.

FIG. 23 is a diagram illustrating an example of the configuration and the control corresponding to the fourth measurement example.

FIG. 24 is a diagram illustrating a first change of information by the biological information measurement system.

FIG. 25 is a diagram illustrating a display example of information after the change by the biological information measurement system.

FIG. 26 is a diagram illustrating a score correction example of the biological information measurement system.

FIG. 27 is a diagram illustrating a second change of information by the biological information measurement system.

FIG. 28 is a diagram illustrating a third change of information by the biological information measurement system.

### DESCRIPTION OF EMBODIMENTS

Hereinafter, embodiments of a biological information measurement system and a toilet seat device disclosed in the present application will be described in detail with reference to the accompanying drawings. Note that the present invention is not limited by the following embodiments. In the present application, a gas that is derived from intestinal fermentation and indicates a high level of health is referred to as a health-related gas, and a gas that is derived from intestinal putrefaction and indicates a low level of health is referred to as an odoriferous gas.

For example, the health-related gas is a gas generated by fermentation by good bacteria in the intestine. For example, the health-related gas may be a gas that is derived from the intestinal fermentation and increases as the level of health in the intestine increases. Specific examples of the health-related gas include hydrogen, carbon dioxide, acetic acid, methane, ethanol, and water.

In addition, for example, the odoriferous gas is a gas generated by fermentation by bad bacteria in the intestine. For example, the odoriferous gas may be a gas containing a sulfur component among fecal gases. Examples of the odoriferous gas include ammonia, trimethylamine, hydrogen sulfide, methyl mercaptan, indole, and skatole. The fecal gas described here is a gas that comes out from the intestine, and for example, the fecal gas includes a gas that comes out during defecation and a gas that is not emitted during the defecation.

### 1. Embodiment

Hereinafter, an outline of a toilet room R as a gas collection place and a biological information measurement system 1 will be described, and then various steps of processing executed by the biological information measurement system 1 and a configuration for executing the steps of processing will be described.

### 1-1. Configuration Example of Toilet Room

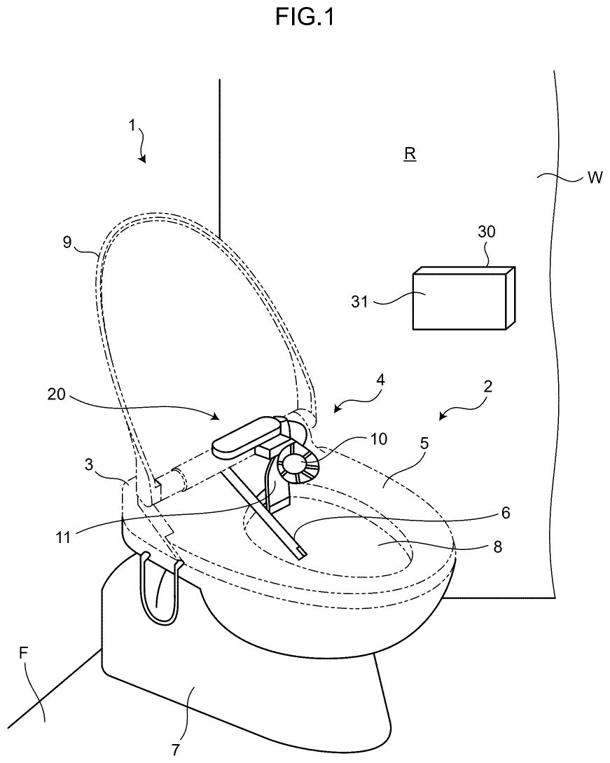

First, a configuration of a biological information measurement system according to an embodiment will be described with reference to FIG. 1. FIG. 1 is a perspective view illustrating an example of a configuration of the biological information measurement system according to the embodiment; In FIG. 1, a measurement device 4 is illustrated in a see-through representation through a toilet seat 5 and a toilet lid 9 in order to illustrate a configuration of the measurement device 4.

As illustrated in FIG. 1, a toilet bowl 7 is installed on a floor surface F in the toilet room R. Hereinafter, a direction facing a space of the toilet room R from the floor surface F may be referred to as an upper side. Components of the biological information measurement system 1 such as the measurement device 4 that performs gas detection and includes a suction device 10 and a gas detection device 20 are disposed in the toilet room R.

The toilet bowl 7 is a closet bowl, and a bowl portion 8 is formed in the toilet bowl 7. The bowl portion 8 has a shape recessed downward and is a portion that receives excreta of a user. The toilet bowl 7 is not limited to a floor-mounted type as illustrated, and may be of any type as long as the biological information measurement system 1 can be applied, such as a wall-mounted type. The toilet bowl 7 is provided with a rim portion over an entire circumference of an end portion of an opening that the bowl portion 8 faces. In the toilet room R, for example, a cleaning water tank for storing cleaning water may be installed in the vicinity of the toilet bowl 7, or a so-called tank-less type in which no cleaning water tank is installed may be used.

For example, when a cleaning operation unit (not illustrated) for cleaning provided in the toilet room R is operated by the user, cleaning water is supplied to the bowl portion 8 of the toilet bowl 7, thereby performing toilet bowl cleaning. The cleaning operation unit may be an operation lever, or a touch operation may be made on a toilet bowl cleaning object displayed on an operation device 30. The cleaning operation unit is not limited to one that is manually operated by the user to perform the toilet bowl cleaning like the operation lever, and may be one that senses a human body using a sensor that senses the user like a seating sensor to perform the toilet bowl cleaning.

A toilet seat device 2 is attached to an upper portion of the toilet bowl 7, and includes a main body portion 3, the measurement device 4, the toilet seat 5, and a cleaning nozzle 6. The toilet seat device 2 is placed on the upper portion of the toilet bowl 7 in which the bowl portion 8 for receiving the excreta is formed. The toilet seat device 2 is placed on the upper portion of the toilet bowl 7 so as to be extended to the bowl portion 8 before the cleaning nozzle 6 sprays cleaning water. The toilet seat device 2 may be detachably attached to the toilet bowl 7 or may be integrally attached to the toilet bowl 7.

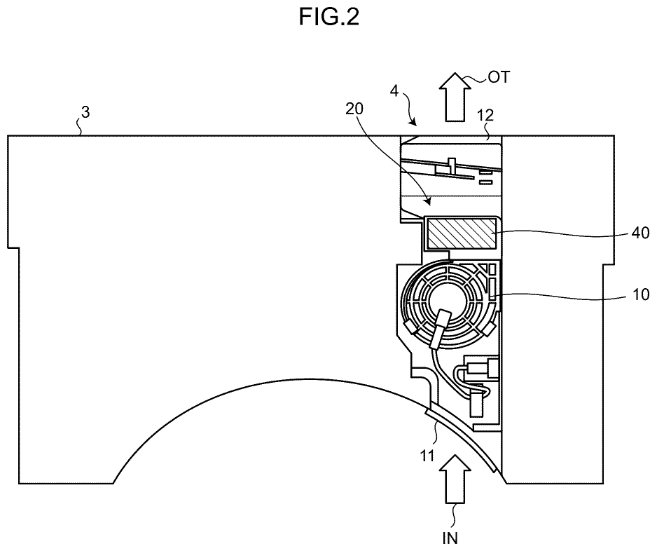

The toilet seat device 2 measures biological information of the user in the toilet room R based on the fecal gas emitted into the bowl portion 8 of the toilet bowl 7 installed in the toilet room R by the configuration of the measurement device 4 and the like. The measurement device 4 includes the suction device 10 and the gas detection device 20. The measurement device 4 will be described in detail with reference to FIG. 2.

As illustrated in FIG. 1, the toilet seat 5 is formed in an annular shape, and is disposed at a position overlapping the opening of the toilet bowl 7 along an end portion (rim portion) of the bowl portion 8. The user is seated on the toilet seat 5. The toilet seat 5 functions as a seating portion that supports the buttocks of the seated user. The toilet lid 9 is attached to the toilet seat device 2 as necessary, and the toilet seat device 2 does not have to include the toilet lid 9.

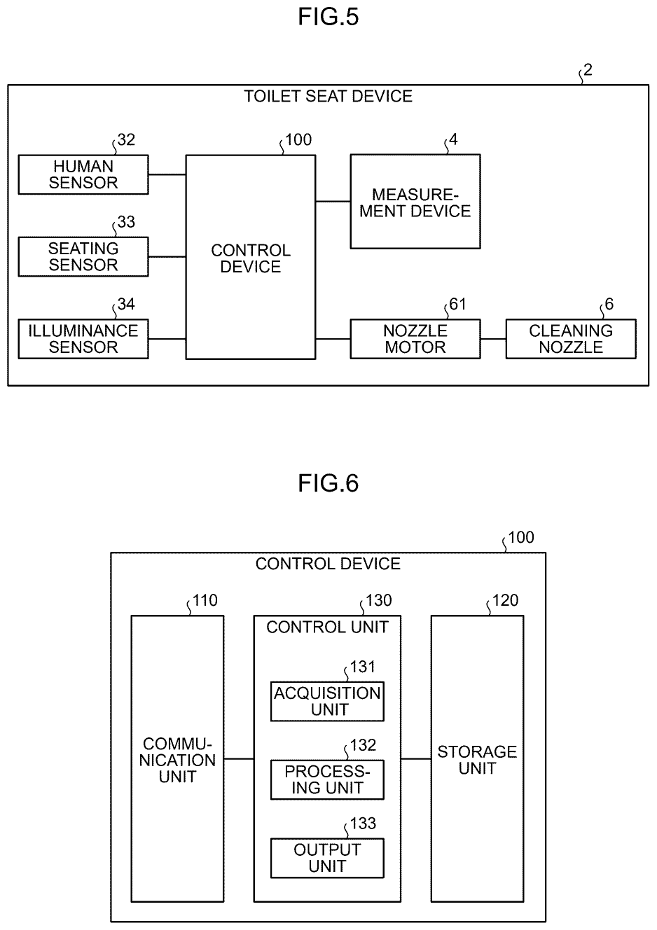

The cleaning nozzle 6 is a nozzle for ejecting water for cleaning. The cleaning nozzle 6 is configured to be movable forward and backward with respect to a housing of the main body portion 3 by driving of a drive source (such as a nozzle motor 61 in FIG. 5) such as an electric motor. The cleaning nozzle 6 is connected to a water source such as a water pipe (not illustrated). As illustrated in FIG. 1, when the cleaning nozzle 6 is at a position (also referred to as “extended position”) at which the cleaning nozzle 6 is extended with respect to the housing of the main body portion 3, the cleaning nozzle 6 sprays the water from the water source to the body of the user to clean the private part.

FIG. 1 illustrates a state in which the cleaning nozzle 6 is at the extended position. The cleaning nozzle 6 may also be used for cleaning the inside of the toilet bowl 7 (the bowl portion 8 or the like). The cleaning nozzle 6 may be used so as to be able to switch between a private part cleaning mode for cleaning the private part of the user and a toilet bowl cleaning mode for spraying the water into the toilet bowl 7. For example, the cleaning nozzle 6 may be used so as to be able to switch between the private part cleaning mode and the toilet bowl cleaning mode under the control of the toilet seat device 2.

The operation device 30 is provided in the toilet room R. The operation device 30 is provided at a position where the user can operate the operation device 30. The operation device 30 is provided at a position where the user can operate the operation device 30 when being seated on the toilet seat 5. In FIG. 1, the operation device 30 is disposed on a wall surface W on a left side when viewed from the user seated on the toilet seat 5. The operation device 30 is not limited to being disposed on the wall surface and may be disposed in various modes as long as the user seated on the toilet seat 5 can use the operation device 30. For example, the operation device 30 may be provided integrally with the toilet seat device 2.

The operation device 30 is communicably connected to the toilet seat device 2 via a predetermined network in a wired or wireless manner. For example, the toilet seat device 2 and the operation device 30 may be connected to each other in any manner as long as information can be transmitted and received, and may be connected to each other in a wired or wireless manner.

The operation device 30 receives various operations from the user via a display surface (for example, a display screen 31) using a touch panel function, for example. Furthermore, the operation device 30 may include a switch or a button, and may receive various operations through the switch, the button, or the like. The display screen 31 is a display screen of a tablet terminal or the like implemented by, for example, a liquid crystal display or an organic electro-luminescence (EL) display, and is a display device for displaying various types of information. That is, the operation device 30 receives an input of the user through the display screen 31 and also performs output to the user. The display screen 31 is a display device that displays various types of information.

The operation device 30 receives a user operation for controlling various functions provided in the toilet room R. The operation device 30 receives a user operation for controlling execution of private part cleaning by the toilet seat device 2. For example, the operation device 30 may include a switch, a button, or the like that receives the user operation described above, and executes various steps of processing according to a user contact with the switch, the button, or the like. The above is an example, and the operation device 30 may receive an operation by the user for executing various steps of processing.

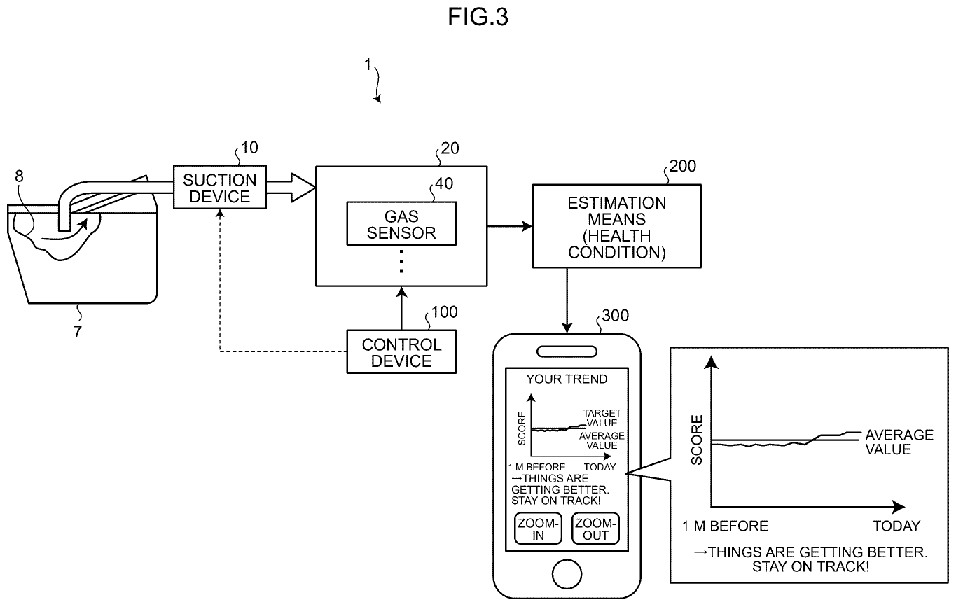

The biological information measurement system 1 measures the biological information of the user in the toilet room R based on the fecal gas emitted into the bowl portion 8 of the toilet bowl 7 installed in the toilet room R by various configurations and processing described below. The biological information measurement system 1 performs control to appropriately measure the fecal gas. The biological information measurement system 1 may provide information to a user terminal (corresponding to display means 300 in FIG. 3) such as a smartphone of the user based on information collected by measurement or the like. In addition, the biological information measurement system 1 may provide information to the operation device 30 (or the display screen 31) of the toilet room R based on the information collected by the measurement or the like.

### 1-2. Configuration of Measurement Device

Next, a configuration of the measurement device 4 will be described with reference to FIG. 2. FIG. 2 is a plan view illustrating an example of the configuration of the measurement device according to the embodiment. In the example illustrated in FIG. 2, a case where the measurement device 4 is disposed in the main body portion 3 is illustrated as an example. In FIG. 2, the configuration of the measurement device 4 is illustrated by removing a portion of the housing (cover) of the main body portion 3 at a position where the measurement device 4 is disposed.

The measurement device 4 includes the suction device 10 that sucks a gas in the bowl portion 8 of the toilet bowl 7 and the gas detection device 20 that detects a component of the sucked gas.

The suction device 10 includes a fan for sucking the gas in the bowl portion 8 of the toilet bowl 7. The suction device 10 is connected to a duct 11 communicating with the inside of the bowl portion 8 of the toilet bowl 7. The duct 11 functions as a flow path for allowing the gas in the bowl portion 8 to flow into the measurement device 4. The suction device 10 drives the fan to suck the gas in the bowl portion 8 by using the duct 11 as the flow path. For example, the suction device 10 executes processing related to the suction under the control of a control device 100. In a case where the suction device 10 doubles as a deodorizing device or the like incorporated in the toilet seat device 2, the suction device 10 may be controlled by control means (device) different from the control device 100.

The gas detection device 20 executes processing related to detection of the component of the gas sucked by the suction device 10. In FIG. 2, the gas detection device 20 is disposed behind the suction device 10 when viewed from the bowl portion 8. FIG. 2 is merely an example, and the gas detection device 20 may be disposed at any position as long as the gas sucked by the suction device 10 can be introduced. The gas detection device 20 is connected to a duct 12 communicating with the outside of the main body portion 3. The duct 12 functions as a flow path for allowing the gas in the gas detection device 20 to flow out from the measurement device 4. For example, the gas in the gas detection device 20 is discharged to the outside of the measurement device 4 using the duct 12 as the flow path in response to driving of the suction device 10.

For example, the gas detection device 20 executes processing related to the gas detection under the control of the control device 100. The gas detection device 20 includes a gas sensor 40 that reacts with a gas contained in a gas mixture. The gas sensor 40 detects a specific component of the gas.

For example, a semiconductor gas sensor is used as the gas sensor 40. The gas sensor 40 may be a hydrogen gas sensor capable of detecting hydrogen. The gas sensor 40 may be an odoriferous gas sensor capable of detecting the odoriferous gas. The gas sensor 40 may be a methane gas sensor capable of detecting methane. For example, the gas detection device 20 includes a plurality of gas sensors 40. The plurality of gas sensors 40 may include a gas sensor 40a that is the hydrogen gas sensor, a gas sensor 40b that is the odoriferous gas sensor, and a gas sensor 40c that is the methane gas sensor. The gas sensors 40a to 40c will be described as the gas sensors 40 unless otherwise specified.

The above is merely an example, and the gas sensor is not limited to the semiconductor gas sensor 40, and a sensor of any aspect may be used. For example, the gas detection device 20 may include one or more arbitrary gas sensors such as a CO₂ sensor, for example, an infrared type carbon dioxide concentration measurement device.

### 1-3. Overall Outline Example of Biological Information Measurement System

Next, an example of an overall outline of the biological information measurement system 1 will be described with reference to FIG. 3. FIG. 3 is a diagram illustrating an example of the overall outline of the biological information measurement system according to the embodiment. A description of points similar to the contents described in FIGS. 1 and 2 will be omitted as appropriate.

In FIG. 3, the biological information measurement system 1 includes the suction device 10, the gas detection device 20, the control device 100, and estimation means 200. FIGS. 1 and 2 illustrate a case where the toilet seat device 2 includes the suction device 10, the gas detection device 20, and the control device 100, but the present disclosure is not limited thereto. For example, the control device 100 may be provided separately from the suction device 10 and the gas detection device 20, and may control the suction device 10 and the gas detection device 20 by communicating with the suction device 10 and the gas detection device 20 in a wireless or wired manner. As described above, the suction device 10 may be controlled by a control unit different from the control device 100.

The estimation means 200 is a computer (information processing device) having a function of executing estimation processing based on information acquired by detection by the gas detection device 20. For example, the estimation means 200 may be a cloud server (server device) positioned outside the toilet room R. In this case, the estimation means 200 is communicably connected to a device (also referred to as “in-toilet device”) disposed in the toilet room R, such as the toilet seat device 2 or the gas detection device 20, via a predetermined network such as the Internet in a wired or wireless manner.

In addition, the estimation means 200 is communicably connected to a device that displays information to the user, such as the display means 300, via a predetermined network in a wired or wireless manner such as the Internet. The estimation means 200 may be communicably connected to the devices such as the in-toilet device and the display means 300 in any manner as long as information can be transmitted and received. The estimation means 200 may be communicably connected in a wired or wireless manner. The estimation means 200 may be communicable with the control device 100.

The estimation means 200 executes the estimation processing regarding a health condition of the user by using information received from the in-toilet device. Data acquired so far may be accumulated in the estimation means 200 or may be accumulated in the display means 300. The estimation means 200 generates information for estimating the health condition of the user (also referred to as “health estimation information”) or information related thereto based on an amount of the health-related gas and an amount of the odoriferous gas in the fecal gas of the user. The estimation means 200 calculates a score as the health estimation information of the user based on a ratio between the amount of the health-related gas and the amount of the odoriferous gas in the fecal gas of the user. For example, the estimation means 200 may use arbitrary information such as the ratio and a single odor. The above is merely an example, and the estimation means 200 may generate arbitrary information as the health estimation information of the user. For example, the estimation means 200 may generate the following information as the health estimation information of the user, or may generate the health estimation information based on the following processing result.

For example, the estimation means 200 may estimate information regarding a state of the intestine of the user from a measurement value. For example, the estimation means 200 may estimate information regarding a state of bacteria. In this case, for example, the estimation means 200 may estimate an occupancy rate of a certain bacterium, an amount and a ratio of good bacteria and bad bacteria, and the like. Furthermore, for example, the estimation means 200 may estimate a state of a metabolite. In this case, for example, the estimation means 200 may estimate amounts of a useful substance and a harmful substance, a ratio thereof, and the like. For example, the estimation means 200 may estimate a state of intestinal pH. In addition, the estimation means 200 may generate information obtained by scoring the above information or evaluating a quality. For example, the estimation means 200 may generate the above information as the health estimation information of the user.

Furthermore, for example, the estimation means 200 may generate information regarding the health condition of the user from the measurement value. In this case, for example, the estimation means 200 may generate a score for an intestinal environment of the user or evaluation information indicating a quality. For example, the estimation means 200 may generate information regarding the intestinal environment of the user. For example, the estimation means 200 may generate information regarding immunity of the user. For example, the estimation means 200 may generate information regarding leanness of the user. For example, the estimation means 200 may generate information regarding a cholesterol index. For example, the estimation means 200 may generate information regarding a metabolic score. For example, the estimation means 200 may generate the above information as the health estimation information of the user. Each example described above is merely an example, and the estimation means 200 is not limited to the above, and may generate various types of information related to the health condition of the user.

The estimation means 200 estimates that the user is healthier as the amount of the health-related gas in the fecal gas of the user is larger than the amount of the odoriferous gas based on the calculated ratio. The estimation means 200 estimates that the user is unhealthier as the amount of the odoriferous gas in the fecal gas of the user is larger than the amount of the health-related gas based on the calculated ratio. The above is merely an example, and the estimation means 200 may perform arbitrary estimation based on the calculated score. The estimation means 200 transmits information to be provided to the user to the display means 300. The estimation means 200 transmits the score calculated as the health estimation information of the user to the display means 300 used by the user.

The estimation means 200 is not limited to the cloud server (server device), and may be an arbitrary device. That is, any form of a device configuration and disposition of the estimation means 200 can be adopted as long as desired processing can be implemented. For example, the estimation means 200 may be a portable terminal (device) such as a notebook computer that can be carried by an administrator or the like of the biological information measurement system 1. Further, the estimation means 200 may be disposed in the toilet room R. For example, the estimation means 200 may be configured to be disposed in the toilet room R. For example, the toilet seat device 2 may have the function of the estimation means 200. In this case, the control device 100 may function as the estimation means 200.

The display means 300 is a display device (computer) that displays information to be provided to the user. For example, the display means 300 may be a user terminal (mobile terminal) owned by the user. In this case, the display means 300 is implemented by, for example, a smartphone, a mobile phone, a personal digital assistant (PDA), a tablet terminal, or a notebook personal computer (PC). For example, the display means 300 is communicably connected to a device included in the biological information measurement system 1 such as the estimation means 200 via a predetermined network in a wired or wireless manner.

The display means 300 transmits and receives information to and from the estimation means 200. The display means 300 receives information to be provided to the user from the estimation means 200. The display means 300 receives the score calculated as the health estimation information of the user from the estimation means 200. The display means 300 displays information including the score calculated as the health estimation information of the user.

In FIG. 3, the display means 300 displays the score calculated as the health estimation information of the user as an intestinal environment score of the user. For example, the display means 300 displays the intestinal environment score of the user in time series for each date and time of excretion. The display means 300 displays a target value of the score, information indicating a temporal change in the intestinal environment score of the user, and text information indicating the evaluation. For example, the display means 300 may request the estimation means 200 for information and display the information acquired from the estimation means 200.

The above is merely an example, and the biological information measurement system 1 can adopt any device configuration as long as desired processing can be implemented. In the biological information measurement system 1, the toilet seat device 2 may have a configuration other than the display means 300. For example, the toilet seat device 2 may include the measurement device 4, the control device 100, and the estimation means 200. Furthermore, for example, the display means 300 does not have to be included in the biological information measurement system 1, or may be included in the biological information measurement system 1. For example, in a case where the display means 300 is the operation device 30 of the toilet room R, the display means 300 may be included in the biological information measurement system 1. In this case, the operation device 30 has a function of displaying the health estimation information of the user.

### 1-4. Action of User and Operation of System

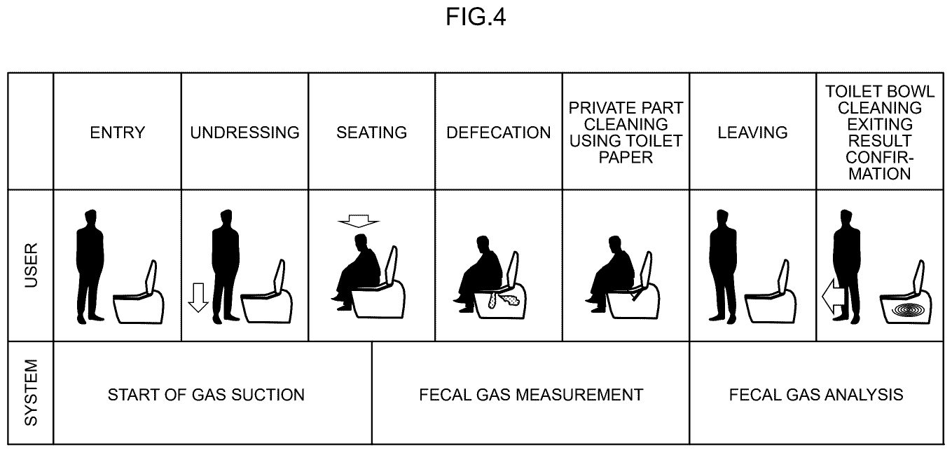

Next, an example of a relationship between a motion (action) of the user who uses the biological information measurement system 1 and a motion (operation) of the biological information measurement system 1 will be described with reference to FIG. 4. FIG. 4 is a diagram illustrating an example of the relationship between the action of the user and the operation of the system.

First, a flow of the action of the user who defecates using the toilet room R will be described with reference to FIG. 4. The user in the toilet room R performs actions of first to seventh stages as illustrated in FIG. 4.

First, the user performs an action of entering the toilet room R as the action of the first stage. The user who has entered the toilet room R performs an action of undressing in the toilet room R as the action of the second stage. The undressed user performs an action of being seated on the toilet seat 5 of the toilet room R as the action of the third stage. The user seated on the toilet seat 5 performs an action of defecating to the bowl portion 8 of the toilet bowl 7 as the action of the fourth stage.

The user who has defecated performs a finishing action such as cleansing the private part after defecation using the private part cleaning function of the toilet seat device 2 or toilet paper as the action of the fifth stage. The user who has completed the finishing after the defecation performs an action of rising and leaving the toilet seat 5 as the action of the sixth stage. The user who has left the seat cleans the toilet bowl 7, exits the toilet room R, confirm a fecal gas analysis result of the biological information measurement system 1, and the like as the action of the seventh stage.

Next, a flow of the operation of the biological information measurement system 1 corresponding to the action of the user described above will be described. The biological information measurement system 1 starts sucking the gas before the user who has entered the toilet room R starts to defecate. In FIG. 4, the biological information measurement system 1 starts the suction of the gas during a period between the first stage and the third stage. Therefore, the biological information measurement system 1 completes measurement preparation before the user defecates. For example, the biological information measurement system 1 sucks the gas mixture (gas) in the bowl portion 8 before the user defecates, thereby sucking a gas serving as a reference (baseline) for comparison with a gas after the defecation of the user. For example, the biological information measurement system 1 calculates an increment (increase amount) from the baseline and estimates (calculates) an amount of the component contained in the fecal gas.

The biological information measurement system 1 measures the fecal gas during a period in which the seated user defecates and leaves the seat. In FIG. 4, the biological information measurement system 1 measures the fecal gas of the user during a period from before the fourth stage to the fifth stage. As a result, the biological information measurement system 1 sucks the gas as needed while the user is seated and acquires data.

The biological information measurement system 1 analyzes the fecal gas after the measurement of the fecal gas is completed. In FIG. 4, the biological information measurement system 1 analyzes the fecal gas of the user during a period between the sixth stage and the seventh stage. As a result, after the user finishes the defecation, the biological information measurement system 1 performs the analysis and calculates the score based on information regarding the fecal gas (result) acquired for the user. The biological information measurement system 1 analyzes the fecal gas of the user and provides the analysis result to the user. The analysis and the provision of the result are not limited to being performed during a period from the sixth stage to the seventh stage, and may be performed at any timing as long as the information can be provided. For example, the biological information measurement system 1 may perform the analysis and provide various types of information such as the result at any timing such as during the measurement or immediately after completion of the measurement.

### 1-5. Functional Configuration of Toilet Seat Device

Next, a functional configuration of the toilet seat device 2 will be described with reference to FIG. 5. FIG. 5 is a block diagram illustrating an example of a configuration of the toilet seat device according to the embodiment. As illustrated in FIG. 5, the toilet seat device 2 includes a human sensor 32, a seating sensor 33, an illuminance sensor 34, the control device 100, a nozzle motor 61, and the cleaning nozzle 6.

The configuration of the toilet seat device 2 illustrated in FIG. 5 is merely an example, and the toilet seat device 2 may include only the toilet seat 5 in a case where each configuration is individually provided. As described above, the configuration of the toilet seat device 2 illustrated in FIG. 5 is merely an example, and any configuration can be adopted as the toilet seat device 2. The human sensor 32, the seating sensor 33, the illuminance sensor 34, and the like may be disposed at arbitrary positions as long as desired sensing is possible. Further, it is sufficient if the toilet seat device 2 can sense seating of the user on the toilet seat 5, and include at least one of the human sensor 32, the seating sensor 33, and the illuminance sensor 34. The toilet seat device 2 transmits and receives information to and from the information processing device such as the estimation means 200 in a wired or wireless manner via a predetermined network (the Internet or the like) by a communication device (for example, a communication unit 110 of the control device 100 in FIG. 6).

The human sensor 32 has a function of sensing a human body. For example, the human sensor 32 is used as seating sensing means that senses seating of the user on the toilet seat 5. For example, the human sensor 32 is implemented by a pyroelectric sensor using an infrared signal. For example, the human sensor 32 may be implemented by a microwave (μ) wave sensor or the like. For example, the human sensor 32 is an infrared projecting/receiving type distance measurement sensor, and may sense the human body present near the toilet seat 5 immediately before the person (user) is seated on the toilet seat 5 or the user seated on the toilet seat 5.

The human sensor 32 also functions as a seat leaving sensor that senses leaving of the user from the toilet seat 5. The human sensor 32 senses a seated state of the user on the toilet seat 5. The human sensor 32 outputs a sensing signal to the control device 100. The above is an example, and the human sensor 32 is not limited thereto and may sense the human body by various means. For example, the human sensor 32 senses a person (the user or the like) who approaches the toilet seat 5.

The seating sensor 33 has a function of sensing seating of a person on the toilet seat device 2. For example, the seating sensor 33 is used as the seating sensing means that senses seating of the user on the toilet seat 5. For example, the seating sensor 33 is implemented by a load sensor or the like. The seating sensor 33 senses that the user is seated on the toilet seat 5. The seating sensor 33 can sense seating of the user on the toilet seat 5.

The seating sensor 33 also functions as the seat leaving sensor that senses leaving of the user from the toilet seat 5. The seating sensor 33 senses the seated state of the user on the toilet seat 5. The above is an example, and the seating sensor 33 is not limited thereto and may sense seating of a person on the toilet seat device 2 by various means. The seating sensor 33 outputs a seating sensing signal to the control device 100.

The illuminance sensor 34 is a sensor that senses an illuminance. For example, the illuminance sensor 34 is used as the seating sensing means that senses seating of the user on the toilet seat 5. For example, the illuminance sensor 34 is disposed at a position facing the bowl portion 8, and senses the illuminance in the bowl portion 8.

The illuminance sensor 34 also functions as the seat leaving sensor that senses leaving of the user the toilet seat 5. The illuminance sensor 34 senses the seated state of the user on the toilet seat 5. The above is merely an example, and the illuminance sensor 34 may be disposed at any position as long as seating of the user on the toilet seat 5 can be sensed by the illuminance.

The control device 100 controls various configurations and processing. The control device 100 is a computer (information processing device) that executes various steps of information processing related to the measurement of the gas and the like. The control device 100 may be any device as long as the device has a configuration necessary for control, and may be, for example, a microcomputer.

The control device 100 controls various configurations for measuring the gas. For example, the control device 100 controls various valves such as a switching valve and a closing valve. For example, the control device 100 controls the switching valve to control a flow path through which the gas flows. For example, the control device 100 switches the flow path through which the gas flows by switching the switching valve. The control device 100 controls the gas detection device 20.

The control device 100 controls the gas detection device 20 to start or stop fecal gas measurement according to the use of the toilet room R by the user. For example, the control device 100 instructs the gas detection device 20 to start the fecal gas measurement when the user is seated on the toilet seat 5, and instructs the gas detection device 20 to stop the fecal gas measurement when the user leaves the toilet seat 5.

The control device 100 transmits control information to the gas detection device 20 in a wired manner. The control device 100 may transmit the control information to the gas detection device 20 in a wireless manner. For example, in a case where the control device 100 is configured as a device separate from the toilet seat device 2, the control device 100 may wirelessly transmit the control information of the gas detection device 20 to the toilet seat device 2. In this case, the control device of the toilet seat device 2 may control the gas detection device 20 based on the received control information.

The control device 100 may control the suction device 10. For example, the control device 100 controls start and stop of the suction by the suction device 10. The control device 100 transmits control information to the suction device 10 in a wired manner. The control device 100 may transmit the control information to the suction device 10 in a wireless manner. For example, in a case where the control device 100 is configured as a device separate from the toilet seat device 2, the control device 100 may wirelessly transmit the control information of the suction device 10 to the toilet seat device 2. In this case, the control device of the toilet seat device 2 may control the suction device 10 based on the received control information.

In addition to the above, the control device 100 controls various configurations of the biological information measurement system 1. The control device 100 controls the nozzle motor 61 and the like. The control device 100 controls the nozzle motor 61 and the like based on a signal transmitted from the operation device 30.

The control device 100 controls the nozzle motor 61 based on a signal of a control instruction related to the private part cleaning transmitted from the operation device 30. The control device 100 controls the nozzle motor 61 to move the cleaning nozzle 6 forward and backward. The control device 100 may control various mechanisms in addition to the nozzle motor 61. For example, the control device 100 controls opening and closing of an electromagnetic valve having a function of a valve that controls a flow of a fluid by an electromagnetic method. For example, the control device 100 switches between supply and stop of tap water from a water supply pipe, for example, by controlling the electromagnetic valve.

The control device 100 transmits control information to the nozzle motor 61 and the like in a wired manner. The control device 100 may transmit the control information to the nozzle motor 61 and the like in a wireless manner. For example, in a case where the control device 100 is configured as a device separate from the toilet seat device 2, the control device 100 may wirelessly transmit the control information of the nozzle motor 61 and the like to the toilet seat device 2. In this case, the control device of the toilet seat device 2 may control the nozzle motor 61 and the like based on the received control information.

Further, the control device 100 may control the toilet lid 9 and the toilet seat 5 as illustrated in FIG. 1. In this case, the control device 100 controls the toilet lid 9 and the toilet seat 5 based on a signal transmitted from the operation device 30. The control device 100 controls the toilet lid 9 based on a signal of a control instruction related to opening and closing of the toilet lid transmitted from the operation device 30. The control device 100 controls the toilet seat 5 based on a signal of a control instruction related to opening and closing of the seating portion transmitted from the operation device 30. The control device 100 transmits control information to the toilet lid 9 and the toilet seat 5 in a wired manner. The control device 100 may transmit the control information to the toilet lid 9 and the toilet seat 5 in a wireless manner.

The control device 100 determines whether or not the seating of the user has been sensed by the seating sensing means such as the human sensor 32, the seating sensor 33, or the illuminance sensor 34. The control device 100 determines whether or not the seating of the user on the toilet seat 5 has been sensed based on the defecation act use prediction information based on the sensing by the seating sensing means acquired from the seating sensing means.

The nozzle motor 61 is the drive source (motor) that drives the cleaning nozzle 6 forward and backward. The nozzle motor 61 performs control to move the cleaning nozzle 6 forward and backward with respect to the main body portion 3. The nozzle motor 61 performs control to move the cleaning nozzle 6 forward and backward according to an instruction from the control device 100.

In the configuration illustrated in FIG. 5, a configuration in which the control device 100 and the like are included in the toilet seat device 2 is illustrated as an example. However, the control device 100, the human sensor 32, the seating sensor 33, the illuminance sensor 34, and the like may be configured as devices separate from the toilet seat device 2. For example, the control device 100 may be configured as a device separate from the toilet seat device 2. For example, the control device 100 may be a server device, and may be disposed at a position separated from the toilet seat device 2. In this case, the control device 100 communicates with each device such as the toilet seat device 2, the human sensor 32, the seating sensor 33, or the illuminance sensor 34, and receives various types of information from each device. In this case, the toilet seat device 2 may have a configuration (a control circuit or the like) for controlling various configurations of the toilet seat device 2 such as the nozzle motor 61. The above is merely an example, and the biological information measurement system 1 can adopt any device configuration as long as desired processing can be implemented.

### 1-6. Functional Configuration of Control Device

Hereinafter, a functional configuration of the control device will be described with reference to FIG. 6. FIG. 6 is a block diagram illustrating an example of a configuration of the control device according to the embodiment. As illustrated in FIG. 6, the control device 100 includes the communication unit 110, a storage unit 120, and a control unit 130. The configuration of the control device 100 is not limited to the configuration illustrated in FIG. 6, and may be another configuration as long as desired processing can be implemented. For example, the control device 100 does not have to include the communication unit 110.

The communication unit 110 is implemented by, for example, a communication circuit or the like. The communication unit 110 is connected to a predetermined network in a wired or wireless manner, and transmits and receives information to and from an external information processing device. For example, the communication unit 110 is connected to a predetermined network in a wired or wireless manner, and transmits and receives information to and from another device such as the operation device 30. The communication unit 110 may be configured as a device (communication device) separate from the control device 100, and may be included in the toilet seat device 2.

The storage unit 120 is implemented by, for example, a semiconductor memory element such as a random access memory (RAM) or a flash memory, or a storage device such as a hard disk or an optical disk. For example, the storage unit 120 is a computer-readable recording medium that non-temporarily records data and the like used by various information processing programs and the like.

The storage unit 120 according to the embodiment stores various types of information necessary for processing. The storage unit 120 stores various types of information acquired from other devices such as various sensors. The storage unit 120 stores various types of information used in various steps of information processing. The storage unit 120 stores information used for various steps of processing. For example, the storage unit 120 stores information regarding a threshold used for processing, such as a first threshold and a second threshold.

The storage unit 120 stores information indicating a predetermined condition used for determining whether or not information can be changed. The storage unit 120 stores information indicating a predetermined condition including at least one of a condition that a first calculation value or a second calculation value is larger than the first threshold and a condition that a third calculation value is smaller than the second threshold smaller than the first threshold. The storage unit 120 stores information indicating a predetermined condition including a condition that the second calculation value exceeds a 0-th calculation value corresponding to the odoriferous gas and a hydrogen gas calculated based on a detection result of a second gas sensor. The storage unit 120 functions as storage means that stores past first information. The storage unit 120 stores various types of history information such as a result of past estimation processing and information output in the past.

Returning to FIG. 6, the description will be continued. The control unit 130 is implemented by, for example, a micro processing unit (MPU), a central processing unit (CPU), or the like executing a program (for example, a program and the like for various steps of information processing according to the present disclosure) stored in the control device 100 using a RAM or the like as a work area. Furthermore, the control unit 130 may be implemented by, for example, an integrated circuit such as an application specific integrated circuit (ASIC) or a field programmable gate array (FPGA).

As illustrated in FIG. 6, the control unit 130 includes an acquisition unit 131, a processing unit 132, and an output unit 133, and implements or executes a function and an action of information processing described below. The internal configuration of the control unit 130 is not limited to the configuration illustrated in FIG. 6, and may be another configuration as long as information processing described below is executed.

The acquisition unit 131 acquires various types of information. The acquisition unit 131 acquires various types of information from the storage unit 120. The acquisition unit 131 receives information from another device. The acquisition unit 131 receives, from various sensors, information (sensing information and the like) sensed by various sensors.

The acquisition unit 131 acquires, from the seating sensing means, information (sensing information and the like) sensed by the seating sensing means. The acquisition unit 131 receives information (sensing information or the like) sensed by at least one of the human sensor 32, the seating sensor 33, and the illuminance sensor 34 from the sensor.

The acquisition unit 131 acquires the defecation act use prediction information based on the sensing by the seating sensing means. For example, the acquisition unit 131 acquires the defecation act use prediction information indicating the seating of the user.

The processing unit 132 performs various steps of processing. The processing unit 132 performs various steps of processing using the information stored in the storage unit 120. The processing unit 132 controls the gas detection device 20.

The processing unit 132 executes determination processing. The processing unit 132 executes the determination processing by using various types of information stored in the storage unit 120. The processing unit 132 determines whether or not to perform the reference value control by using various types of information acquired by the acquisition unit 131.

The processing unit 132 executes calculation processing. The processing unit 132 executes the calculation processing by using various types of information stored in the storage unit 120. The processing unit 132 executes the calculation processing by using various types of information acquired by the acquisition unit 131.

The processing unit 132 calculates various types of information regarding the gas. The processing unit 132 calculates a value based on the measurement value measured by the gas detection device 20. The processing unit 132 calculates a resistance value of a sensor element based on a voltage value measured by the gas sensor 40. For example, the processing unit 132 calculates the resistance value of the sensor element from the measured voltage value by using a function indicating a relationship between the voltage value and the resistance value of the sensor element. The processing unit 132 calculates a reciprocal (also referred to as a “calculation value”) of the resistance value of the sensor element using Expression (1).

The processing unit 132 may calculate a concentration of the gas based on the calculated resistance value of the sensor element. In this case, the processing unit 132 calculates the concentration of the gas from the calculated resistance value by using a function indicating a relationship between the resistance value and the concentration of the gas.

The processing unit 132 executes the estimation processing of estimating information (health estimation information) for estimating the health condition of the user or information related thereto based on the first calculation value corresponding to the hydrogen gas and the third calculation value corresponding to the odoriferous gas obtained based on a detection result of the gas sensor 40a serving as a first gas sensor. In a case where the estimation means 200 executes the estimation processing, the processing unit 132 does not have to execute the estimation processing.

The processing unit 132 calculates the second calculation value corresponding to the hydrogen gas of the gas sensor 40b based on a plurality of calculation values corresponding to the hydrogen gas contained in the gas mixture. The processing unit 132 calculates the third calculation value corresponding to the odoriferous gas based on a detection result of the gas sensor 40b and the second calculation value. The processing unit 132 calculates the second calculation value by using a plurality of calculation values including the first calculation value corresponding to the hydrogen gas obtained based on the detection result of the gas sensor 40a.

The processing unit 132 calculates the second calculation value that is a statistical value of a plurality of calculation values obtained by measuring the gas in a sealed space a plurality of times using the gas sensor 40a. The processing unit 132 calculates the second calculation value that is a statistical value of a plurality of calculation values obtained by measuring the gas in a storage portion a plurality of times. The processing unit 132 calculates the second calculation value that is a statistical value of one or more calculation values obtained by measuring the gas in the flow path once or a plurality of times.

The processing unit 132 calculates the second calculation value by using a plurality of calculation values including the first calculation value and a fourth calculation value corresponding to the hydrogen gas obtained based on a detection result of the gas sensor 40c. For example, in a case where a difference between an amount or concentration obtained from the first calculation value and an amount or concentration obtained from the fourth calculation value is equal to or smaller than a predetermined value, the processing unit 132 determines that the methane gas is not contained in the fecal gas of the user. For example, in a case where the difference between the amount or concentration obtained from the first calculation value and the amount or concentration obtained from the fourth calculation value is larger than the predetermined value, the processing unit 132 determines that the methane gas is contained in the fecal gas of the user.

In a case where it is determined that the methane gas is contained in the fecal gas of the user, the processing unit 132 uses the gas sensor 40c as a sensor for detecting the methane gas. In a case where it is determined that the methane gas is not contained in the fecal gas of the user, the processing unit 132 uses the gas sensor 40c as a sensor for detecting the hydrogen gas.

The processing unit 132 calculates the first calculation value corresponding to the hydrogen gas based on the detection result of the gas sensor 40a serving as the first gas sensor. The processing unit 132 calculates the second calculation value corresponding to the hydrogen gas of the gas sensor 40b serving as the second gas sensor based on the first calculation value. In a case where the gas sensor 40c is used as the sensor for detecting the hydrogen gas, the processing unit 132 calculates the second calculation value based on the first calculation value and the fourth calculation value. The processing unit 132 calculates the third calculation value corresponding to the odoriferous gas based on the detection result of the gas sensor 40b and the second calculation value.

The processing unit 132 performs correction on at least one of the 0-th calculation value, the second calculation value, or the third calculation value corresponding to the odoriferous gas and the hydrogen gas calculated based on the detection result of the gas sensor 40b. As the correction, the processing unit 132 performs correction of decreasing the second calculation value or increasing the 0-th calculation value.

The processing unit 132 performs the correction in a case where the first calculation value or the second calculation value is larger than the first threshold or in a case where the third calculation value is smaller than the second threshold smaller than the first threshold. The processing unit 132 performs the correction in a case where the second calculation value is larger than the 0-th calculation value. The processing unit 132 has a correction value set in advance as a value corresponding to the odoriferous gas, and replaces the third calculation value with the correction value as the correction in a case where the third calculation value is smaller than a third threshold.

In a case where at least one of the first calculation value, the second calculation value, and the third calculation value satisfies a predetermined condition, the processing unit 132 performs control to change the first information, which is the health condition of the user or information regarding the health condition of the user output by output means, regardless of the third calculation value. In a case where the predetermined condition is satisfied, the processing unit 132 changes a value included in the first information to a preset setting value.

The processing unit 132 changes the first information based on the past first information stored in the storage means. In a case where the predetermined condition is satisfied, the processing unit 132 determines to output second information regarding measurement accuracy. In a case where the predetermined condition is satisfied, the processing unit 132 determines to output third information regarding a measurement error.

The output unit 133 performs output processing of outputting various types of information. The output unit 133 functions as a transmission unit that transmits various types of information. The output unit 133 performs the output processing by transmitting information to an external information processing device. The output unit 133 transmits information to the external information processing device. For example, the output unit 133 transmits various types of information to the estimation means 200. For example, the output unit 133 transmits various types of information to an administrator device such as a personal computer or a smartphone used by an administrator of the estimation means 200. Furthermore, the output unit 133 may perform the output processing by transmitting information to the operation device 30 (or the display screen 31).

The output unit 133 transmits various types of information used for the estimation processing by the estimation means 200 to the estimation means 200. The output unit 133 transmits information indicating the measurement value acquired by the gas detection device 20. The output unit 133 transmits information indicating the calculation value calculated by the processing unit 132.

The output unit 133 outputs each piece of information such as a content. In a case where the predetermined condition is satisfied, the output unit 133 outputs information including a changed score obtained by changing the score that is original data. In a case where the predetermined condition is not satisfied, the output unit 133 outputs information including the score that is the original data. In a case where the predetermined condition is satisfied, the output unit 133 outputs the second information regarding the measurement accuracy. In a case where the predetermined condition is satisfied, the output unit 133 outputs the third information regarding the measurement error.

### 1-7. Gas Sensor

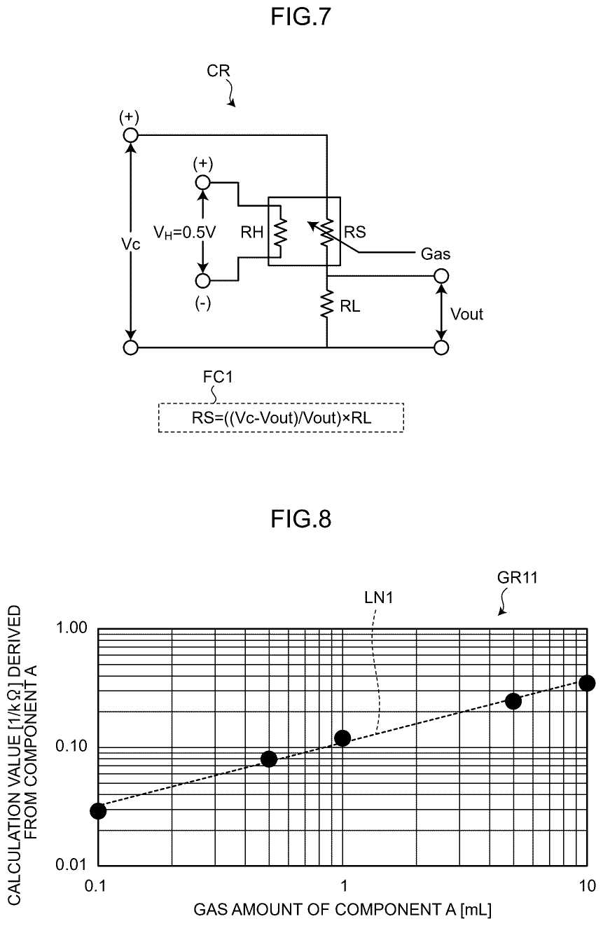

Hereinafter, a configuration example of the gas sensor will be described with reference to FIG. 7. FIG. 7 is a diagram illustrating an example of a configuration of the gas sensor. Specifically, FIG. 7 is a diagram illustrating an example of a circuit configuration CR of the semiconductor gas sensor 40.

The gas sensor 40 includes the sensor element and the resistor element for measurement. In FIG. 7, the gas sensor 40 has the circuit configuration CR in which the sensor element (corresponding to a sensor resistor RS in FIG. 7) and the resistor element for measurement (corresponding to a resistor element RL in FIG. 7) are connected in series.

In the semiconductor gas sensor 40, a value related to a gas amount is calculated using the following Expression (1). Expression (1) corresponds to the circuit configuration CR illustrated in FIG. 7, and is an expression similar to a function FC1 in FIG. 7.

$$RS = ( ( Vc - Vout ) / ( Vout ) \times RL \qquad ( 1 )$$

“RS” in Expression (1) represents the resistance value of the sensor element. For example, “RS” in Expression (1) indicates the resistance value of the sensor resistor RS, which is an example of a value calculated based on measurement by the gas sensor 40. As described above, Expression (1) is a calculation expression of the resistance value.

“RL” in Expression (1) represents the resistance value of the resistor element RL. “Vc” in Expression (1) represents a voltage value of a circuit voltage Vc. “Vout” in Expression (1) represents a voltage value of an output voltage Vout in the resistor element. For example, “Vout” in Expression (1) indicates a voltage value of the resistor element RL, which is an example of the measurement value acquired by the gas sensor 40.

The resistance value of the sensor resistor RS in Expression (1) is an index related to the gas amount. The biological information measurement system 1 calculates the index (resistance value) related to the gas amount from the measurement value (voltage value), and calculates the gas amount from the calculated resistance value. Although the principle and the like of the semiconductor gas sensor will not be described in detail, for example, “RH” illustrated only in the circuit configuration CR of FIG. 7 corresponds to a heater (resistor) for heating the sensor element, and “V_{H}” corresponds to a voltage of the heater. The gas sensor in the present invention is not limited to a semiconductor sensor, and can be substituted by any sensor that satisfies Expression (1).

### 1-8. Outline of Processing in Biological Information Measurement System

Hereinafter, a processing example on the premise of the above-described configuration of the biological information measurement system 1 will be described. First, prior to the description of various steps of processing in the biological information measurement system 1, a relationship between the gas sensor or a value based on the measurement performed by the gas sensor and the amount of the gas will be described. A description of matters similar to the contents described above will be omitted as appropriate.

### 1-8-1. Example of Relationship Between Measurement Performed by Gas Sensor and Amount

First, the relationship between the measurement performed by the gas sensor and the amount will be described with reference to FIG. 8. FIG. 8 is a diagram illustrating an example of a relationship between the value based on the measurement performed by the sensor and the amount of the gas.

For example, FIG. 8 illustrates a graph GR11 that is a double logarithmic graph of a calculation value (the reciprocal of the resistance value) of a component A based on the measurement performed by the gas sensor and a gas amount (also simply referred to as “amount”) of the component A. Specifically, in the graph GR11 in FIG. 8, a vertical axis represents the calculation value “1/kΩ” of the component A, and a horizontal axis represents the amount “mL” of the component A.

A point (∘) in the graph GR11 corresponds to, for example, a result of actual measurement performed for deriving the relationship between the measurement performed by the gas sensor and the amount, and indicates the calculation value of the component A obtained from a result of actual measurement performed by the gas sensor for a gas containing the component A in the amount corresponding to the horizontal axis. Although only five points (actual measurement results) are illustrated in FIG. 8 for the sake of explanation, the number of actual measurement results may be six or more or four or less.

A line LN1 of the graph GR11 indicates the relationship between the calculation value derived from the component A and the gas amount of the component A. An equation (function) corresponding to the line LN1 is a regression equation for calculating (estimating) the gas amount from the calculation value for the component A. For example, the equation (function) corresponding to the line LN1 is derived by regression analysis or the like using the points (actual measurement results) in the graph GR11.

As described above, the calculation value derived from the component A based on the measurement performed by the gas sensor and the amount of the component A both have a linear correlation in a log scale. Therefore, the gas amount of the component A can be calculated from the calculation value such as a peak value of the gas sensor. That is, each gas amount can be calculated from the calculation value based on measurement of each of a plurality of gas sensors. By calculating the gas amount of each component, the biological information measurement system 1 can calculate a ratio relationship between the amount of the health-related gas obtained by adding the gas amounts of the components corresponding to the health-related gas and the amount of the odoriferous gas obtained by adding the gas amounts of the components corresponding to the odoriferous gas.

### 1-8-2. Example of Gas Sensor and Reacting Component

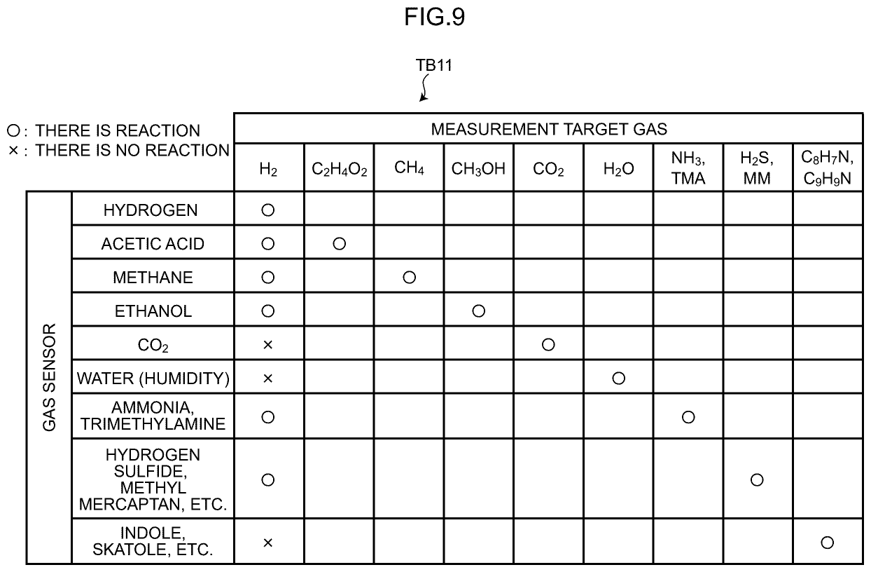

Next, an example of the gas sensor and the reacting component will be described with reference to FIG. 9. FIG. 9 is a diagram illustrating an example of the gas sensor and the reacting component. A correspondence table TB11 of FIG. 9 shows a correspondence relationship between each gas sensor and a component with which the gas sensor reacts. In FIG. 9, a case where the gas sensor reacts with the component is indicated by “∘”, and a case where the gas sensor does not react with the component is indicated by “x”. As shown in the correspondence table TB11 in FIG. 9, the reacting component is different for each gas sensor.

In FIG. 9, the first gas sensor (hydrogen gas sensor) is a sensor that reacts only with hydrogen (H₂). For example, the gas sensor 40a is the first gas sensor (hydrogen gas sensor). For example, the gas sensor 40a of the biological information measurement system 1 is a gas sensor that reacts only with hydrogen, that is, a gas sensor in which the resistance value of the sensor resistor RS in Expression (1) changes according to a change in the amount of hydrogen.

In the case of reacting only with hydrogen like the first gas sensor illustrated in FIG. 9, a calculation value derived from hydrogen contained in the fecal gas of the user is expressed by the following Expression (2).

$$1 / {R}_{s \_ 1} = 1 / {R}_{air} + 1 / {R}_{H 2\_ 1} \qquad ( 2 )$$

“R_{s_1}” in Expression (2) corresponds to the resistance value of the sensor resistor RS of the first gas sensor. For example, “R_{s_1}” in Expression (2) is the resistance value of the sensor resistor RS calculated from the measurement value of the first gas sensor.

“Rₐᵢᵣ” in Expression (2) corresponds to the resistance value derived from the baseline in the first gas sensor. For example, “Rₐᵢᵣ” in Expression (2) is the resistance value derived from the air in the bowl portion 8 before the fecal gas of the user is released.

“R_{H2_1}” in Expression (2) corresponds to the resistance value derived from the hydrogen gas in the first gas sensor. For example, “R_{H2_1}” in Expression (2) is the resistance value derived from hydrogen contained in the fecal gas released from the user.

“R_{s_1}” in Expression (2) and “Rₐᵢᵣ” in Expression (2) are calculation values based on the measurement performed by the first gas sensor, and are detected (acquired) by the measurement performed by the first gas sensor. Therefore, the biological information measurement system 1 calculates “R_{H2_1}” in Expression (2) by substituting the value obtained by the measurement performed by the first gas sensor into Expression (2).

On the other hand, in FIG. 9, the second gas sensor (odoriferous gas sensor) is a sensor that reacts not only with the odoriferous gas (H₂S or the like) but also with hydrogen (H₂). For example, the gas sensor 40b of the biological information measurement system 1 is the second gas sensor (odoriferous gas sensor). For example, the gas sensor 40b is a gas sensor that reacts with the odoriferous gas and hydrogen, that is, a gas sensor in which the resistance value of the sensor resistor RS in Expression (1) changes according to a change in the amount of the odoriferous gas and the amount of hydrogen. In the present embodiment, a component for each detection unit is adjusted such that the detection unit used in the hydrogen gas sensor strongly reacts with the hydrogen gas, and the detection unit used in the odoriferous gas sensor strongly reacts with the odoriferous gas.

In the case of reacting with the odoriferous gas and hydrogen like the second gas sensor illustrated in FIG. 9, the calculation value derived from the odoriferous gas contained in the fecal gas of the user is expressed by the following Expression (3).

$$1 / {R}_{s \_ 2} = 1 / {R}_{air} + 1 / {R}_{H 2\_ 2} + 1 / {R}_{H 2 S \_ 2} \qquad ( 3 )$$

“R_{s_2}” in Expression (3) corresponds to the resistance value of the sensor resistor RS of the second gas sensor. For example, “R_{s_2}” in Expression (3) is the resistance value of the sensor resistor RS calculated from the measurement value of the second gas sensor.

“Rₐᵢᵣ” in Expression (3) corresponds to the resistance value derived from the baseline in the second gas sensor. For example, “Rₐᵢᵣ” in Expression (3) is the resistance value derived from the air in the bowl portion 8 before the fecal gas of the user is released.

“R_{H2_2}” in Expression (3) corresponds to the resistance value derived from the hydrogen gas in the second gas sensor. For example, “R_{H2_2}” in Expression (3) is the resistance value derived from hydrogen contained in the fecal gas released from the user.

“R_{H2S_2}” in Expression (3) corresponds to the resistance value derived from the odoriferous gas in the second gas sensor. For example, “R_{H2S_2}” in Expression (3) is the resistance value derived from an odoriferous gas such as hydrogen sulfide contained in the fecal gas released from the user.

“R_{s_2}” in Expression (3) and “Rₐᵢᵣ” in Expression (3) are the calculation values based on the measurement performed by the second gas sensor, and are detected (acquired) by the measurement performed by the second gas sensor. However, two variables of “R_{H2_2}” and “R_{H2S_2}” are undetermined in Expression (3). Therefore, the biological information measurement system 1 cannot determine a value of “R_{H2S_2}” in Expression (3) only by Expression (3). Therefore, the biological information measurement system 1 calculates (estimates) the amount of the odoriferous gas by using information of a gas sensor other than the second gas sensor, which is described below.

In FIG. 9, a third gas sensor (methane gas sensor) reacts not only with methane (CH₄ or the like) but also with hydrogen (H₂). For example, the gas sensor 40c of the biological information measurement system 1 is the third gas sensor (methane gas sensor). For example, the gas sensor 40c is a gas sensor that reacts with methane and hydrogen, that is, a gas sensor in which the resistance value of the sensor resistor RS in Expression (1) changes according to a change in the amount of methane and the amount of hydrogen.

In FIG. 9, a fourth gas sensor (carbon dioxide gas sensor) is a sensor that reacts only with carbon dioxide (CO₂). For example, the CO₂ sensor of the biological information measurement system 1 is the fourth gas sensor (carbon dioxide gas sensor). For example, the CO₂ sensor is an infrared gas sensor that reacts only with carbon dioxide.

### 1-8-3. Calculation Example of Amount of Odoriferous Gas

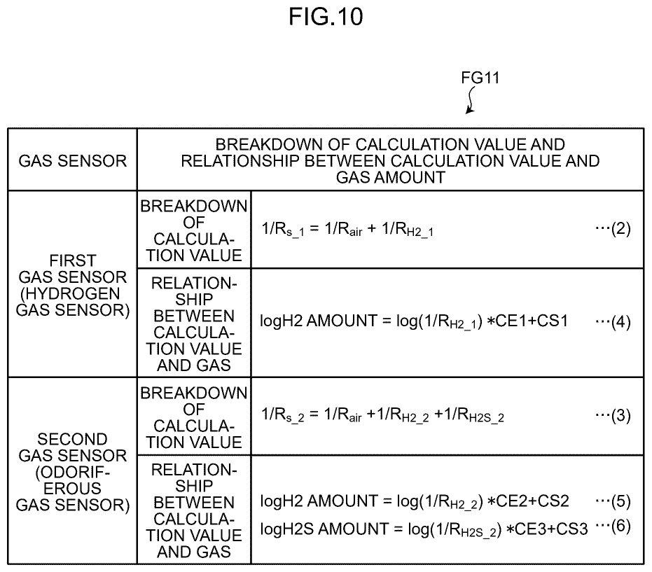

As described above, the calculation value (the reciprocal of the resistance value) of the second gas sensor (odoriferous gas sensor) is the sum of several types of components. For example, the calculation value (the reciprocal of the resistance value) of the second gas sensor is the sum of the calculation value (the reciprocal of the resistance value) of the baseline and the calculation value (the reciprocal of the resistance value) derived from the reacting component (odoriferous gas+health-related gas). Therefore, the biological information measurement system 1 derives the calculation value derived from each component by simultaneous equations based on equations corresponding to the plurality of gas sensors. This matter will be described with reference to FIG. 10. FIG. 10 is a diagram illustrating an example of processing of calculating the amount of the odoriferous gas.

A function group FG11 in FIG. 10 illustrates Expressions (2) to (6) used for calculation (estimation) of the amount of the odoriferous gas and a correspondence relationship thereof.

Expressions (2) and (4) show the breakdown of the calculation values in the first gas sensor (hydrogen gas sensor) and a relationship between the calculation values and the gas amount. Expression (2) in FIG. 10 is the same as Expression (2) described above, and a detailed description thereof will be omitted.

$$log H 2 amount = log ( 1 / {R}_{H 2\_ 1} ) ⋆ CE 1 + CS 1 \qquad ( 4 )$$

The “H2 amount” in Expression (4) is the amount of the hydrogen gas calculated (estimated) by the measurement performed by the first gas sensor, and the “logH2 amount” corresponds to a log expression (logarithmic value) of the amount of the hydrogen gas.

“R_{H2_1}” in Expression (4) is the resistance value derived from the hydrogen gas based on the measurement performed by the first gas sensor, and “log (1/R_{H2_1})” corresponds to a log expression (logarithmic value) of the calculation value (the reciprocal of the resistance value) derived from the hydrogen gas.

“CE1” in Expression (4) is a coefficient related to “log (1/R_{H2_1})”, and an arbitrary value such as “−0.4 . . . ” is set. Furthermore, “CS1” in Expression (4) is a constant to be added to “log (1/R_{H2_1})*CE1”, and an arbitrary value such as “1.1 . . . ” is set. For example, the administrator or the like of the biological information measurement system 1 derives the coefficient “CE1” and the constant “CS1” included in Expression (4) by actual measurement or the like, and sets Expression (4).

Expressions (3), (5), and (6) show the breakdown of the calculation values in the second gas sensor (odoriferous gas sensor) and a relationship between the calculation values and the gas amount. Expression (3) in FIG. 10 is the same as Expression (3) described above, and a detailed description thereof will be omitted.

$$log H 2 amount = log ( 1 / {R}_{H 2\_ 2} ) ⋆ CE 2 + CS 2 \qquad ( 5 )$$

The “H2 amount” in Expression (5) is the amount of the hydrogen gas calculated (estimated) by the measurement performed by the second gas sensor, and the “logH2 amount” corresponds to a log expression (logarithmic value) of the amount of the hydrogen gas.

“R_{H2_2}” in Expression (5) is the resistance value derived from the hydrogen gas based on the measurement performed by the second gas sensor, and “log (1/R_{H2_2})” corresponds to a log expression (logarithmic value) of the calculation value (the reciprocal of the resistance value) derived from the hydrogen gas.

“CE2” in Expression (5) is a coefficient related to “log (1/R_{H2_2})”, and an arbitrary value such as “−0.8 . . . ” is set. Furthermore, “CS2” in Expression (5) is a constant to be added to “log (1/R_{H2_2})*CE2”, and an arbitrary value such as “2.0 . . . ” is set. For example, the administrator or the like of the biological information measurement system 1 derives the coefficient “CE2” and the constant “CS2” included in Expression (5) by actual measurement or the like, and sets Expression (5).

$$log H 2 S amount = log ( 1 / {R}_{H 2 S \_ 2} ) ⋆ CE 3 + CS 3 \qquad ( 6 )$$

The “H₂S amount” in Expression (5) is the amount of the odoriferous gas calculated (estimated) by the measurement performed by the second gas sensor, and the “logH2S amount” corresponds to a log expression (logarithmic value) of the amount of the odoriferous gas.

“R_{H2S_2}” in Expression (5) is the resistance value derived from the odoriferous gas based on the measurement performed by the second gas sensor, and “log (1/R_{H2S_2})” corresponds to a log expression (logarithmic value) of the calculation value (the reciprocal of the resistance value) derived from the odoriferous gas.

“CE3” in Expression (5) is a coefficient related to “log (1/R_{H2S_2})”, and an arbitrary value such as “−0.7 . . . ” is set. In addition, “CS3” in Expression (5) is a constant to be added to “log (1/R_{H2S_2})*CE3”, and an arbitrary value such as “1.8 . . . ” is set. For example, the administrator or the like of the biological information measurement system 1 derives the coefficient “CE3” and the constant “CS3” included in Expression (6) by actual measurement or the like, and sets Expression (6).

Hereinafter, an example of processing of calculating (estimating) the amount of the odoriferous gas by the biological information measurement system 1 using Expressions (2) to (6) will be described.

First, the biological information measurement system 1 obtains a value related to the amount of the hydrogen gas based on the measurement performed by the first gas sensor using Expressions (2) and (4). For example, the biological information measurement system 1 obtains a value of the “logH2 amount” in Expression (4) by using the values (the value of “R_{s_1}” and the value of “Rₐᵢᵣ” in Expression (2)) obtained by the measurement performed by the gas sensor 40a, Expression (2), and Expression (4).

Then, the biological information measurement system 1 applies the obtained value of the “logH2 amount” in Expression (4) to Expression (5), and obtains the value of “R_{H2S_2}” in Expression (5).

Then, the biological information measurement system 1 applies the obtained value of “R_{H2S_2}” in Expression (5) to Expression (3), and obtains the value of “R_{H2S_2}” in Expression (5). For example, the biological information measurement system 1 applies the obtained value of “R_{H2S_2}” in Expression (5) and the values acquired by the measurement performed by the gas sensor 40b (the value of “R_{s_2}” and the value of “Rₐᵢᵣ” in Expression (3)) to Expression (3) and obtains the value of “R_{H2S_2}” in Expression (3). For example, the biological information measurement system 1 applies the obtained value of “R_{H2S_2}” in Expression (3) to Expression (6), and obtains the value of “H₂S amount” in Expression (6).

As described above, the biological information measurement system 1 calculates (estimates) the amount of the odoriferous gas by subtracting an influence derived from the hydrogen gas from an output of the second gas sensor (odoriferous gas sensor). The above-described processing is merely an example, and the biological information measurement system 1 may execute any processing as long as the amount of the odoriferous gas can be calculated (estimated).

### 1-8-4. Problem in Calculation of Amount of Odoriferous Gas

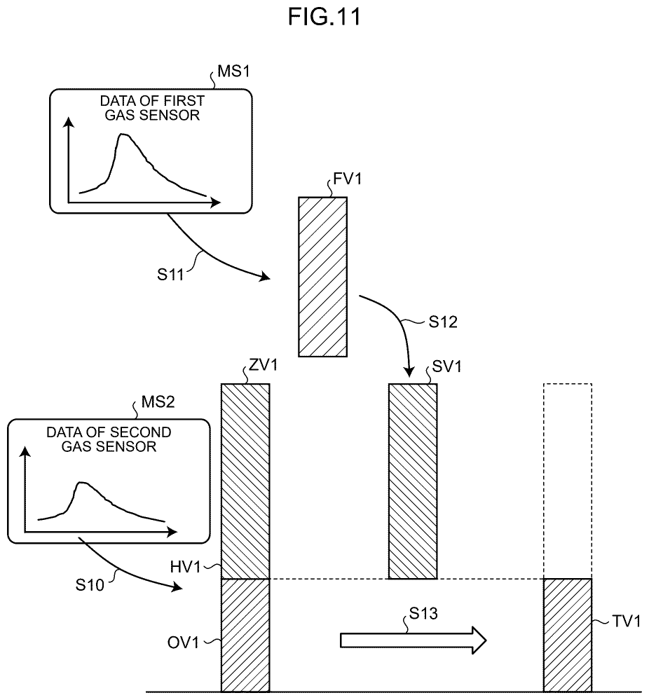

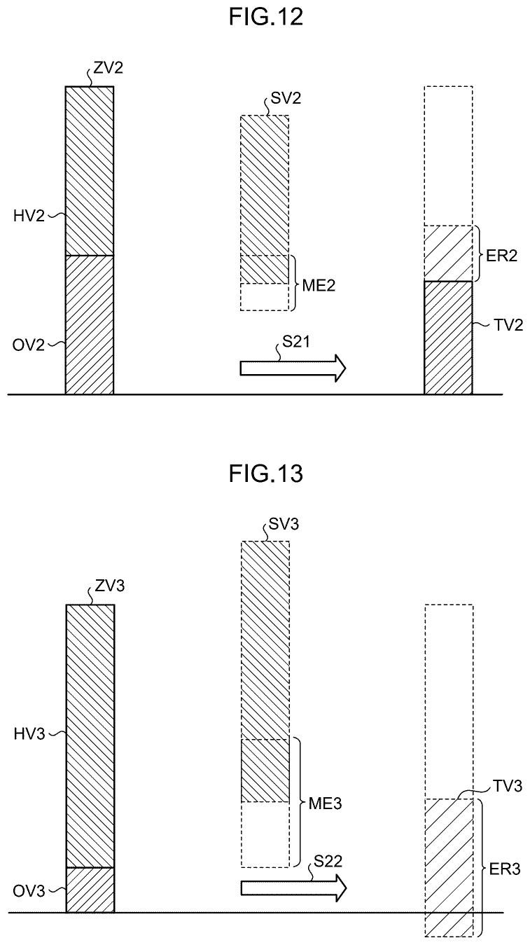

Hereinafter, a problem in the case of calculating the amount of the odoriferous gas using the measurement performed by the hydrogen gas sensor as described above will be described with reference to FIG. 11. FIG. 11 is a diagram illustrating an outline of the calculation of the amount of the odoriferous gas.

Measurement MS2 in FIG. 11 corresponds to the measurement performed by the second gas sensor (odoriferous gas sensor). For example, a waveform in the measurement MS2 indicates a sensor output (for example, a voltage value). In odoriferous gas calculation processing, a 0-th calculation value ZV1 is calculated as the 0-th calculation value corresponding to the hydrogen gas and the odoriferous gas obtained based on a detection result of the measurement MS2 performed by the second gas sensor (Step S10). For example, a length of the 0-th calculation value ZV1 indicates a magnitude of the 0-th calculation value ZV1. The 0-th calculation value ZV1 in FIG. 11 includes a first mixed value HV1 that is a value corresponding to the hydrogen gas and a second mixed value OV1 that is a value corresponding to the odoriferous gas.

For example, the first mixed value HV1 is an accurate value (correct value) corresponding to “R_{H2_2}” in Expression (3). Further, for example, the second mixed value OV1 is an accurate value (correct value) corresponding to “R_{H2S_2}” in Expression (3). In FIG. 11, for the sake of explanation, the breakdown of the first mixed value HV1 and the second mixed value OV1 in the 0-th calculation value ZV1 is illustrated, but the first mixed value HV1 and the second mixed value OV1 are actually estimated based on the third calculation value described below.

Measurement MS1 in FIG. 11 corresponds to the measurement performed by the first gas sensor (hydrogen gas sensor). For example, a waveform in the measurement MS1 indicates a sensor output (for example, a voltage value). In the odoriferous gas calculation processing, a first calculation value FV1 is calculated as the first calculation value corresponding to the hydrogen gas obtained based on a detection result of the measurement MS1 performed by the first gas sensor (Step S11). For example, the first calculation value FV1 corresponds to “R_{H2_1}” in Expression (2). Reference is made to the calculation value related to the amount of the hydrogen gas calculated from the measurement value of the first gas sensor. The numbers in Step S11 and the like are reference numerals for distinguishing and describing the respective steps of processing, and do not indicate the order. For example, Step S11 may be executed before Step S10.

In the odoriferous gas calculation processing, a second calculation value SV1 is calculated as the second calculation value corresponding to the hydrogen gas of the second gas sensor based on the first calculation value FV1 (Step S12). For example, the second calculation value SV1 is an estimated value corresponding to “R_{H2_2}” in Expression (3). The second calculation value derived from the hydrogen gas contained in the second gas sensor is calculated from the first calculation value FV1. For example, in a case where it is determined that the methane gas is not contained in the fecal gas of the user, the processing unit 132 calculates the second calculation value SV1 corresponding to the hydrogen gas of the second gas sensor based on the first calculation value FV1.

In the odoriferous gas calculation processing, a third calculation value TV1 is calculated as the third calculation value corresponding to the odoriferous gas based on the 0-th calculation value ZV1 and the second calculation value SV1 (Step S13). The third calculation value TV1 is calculated as the third calculation value corresponding to the odoriferous gas by subtracting the second calculation value SV1 from the 0-th calculation value ZV1. For example, the third calculation value TV1 is an estimated value corresponding to “R_{H2S_2}” in Expression (3). In the odoriferous gas calculation processing, since the amount of the odoriferous gas is calculated (estimated) using the third calculation value TV1 calculated by the processing as described above, measurement variations of the first gas sensor affect the final calculation (estimation) of the amount of the odoriferous gas. Therefore, in the odoriferous gas calculation processing, there may be a problem in the calculation of the amount of the odoriferous gas.

Hereinafter, a specific example of the problem in the calculation of the amount of the odoriferous gas will be described with reference to FIGS. 12 and 13. FIGS. 12 and 13 are diagrams illustrating an example of the influence of the measurement variations of the gas sensor on the calculation. A description of matters similar to the contents described in FIG. 11 and the like will be omitted as appropriate.

First, a problem that may occur even in a case where a certain amount of odoriferous gas is present will be described with reference to FIG. 12.

A 0-th calculation value ZV2 is the 0-th calculation value corresponding to the hydrogen gas and the odoriferous gas obtained based on the detection result of the measurement performed by the second gas sensor. The 0-th calculation value ZV2 in FIG. 12 includes a first mixed value HV2 that is a value corresponding to the hydrogen gas and a second mixed value OV2 that is a value corresponding to the odoriferous gas.

For example, the first mixed value HV2 is an accurate value (correct value) corresponding to “R_{H2_2}” in Expression (3). Further, for example, the second mixed value OV2 is an accurate value (correct value) corresponding to “R_{H2S_2}” in Expression (3). In FIG. 12, for the sake of explanation, the breakdown of the first mixed value HV2 and the second mixed value OV2 in the 0-th calculation value ZV2 is illustrated, but the first mixed value HV2 and the second mixed value OV2 are actually estimated based on the third calculation value described below.

A second calculation value SV2 in FIG. 12 is calculated based on the first calculation value corresponding to the hydrogen gas obtained based on the detection result of the measurement performed by the first gas sensor. The second calculation value SV2 is the second calculation value corresponding to the hydrogen gas of the second gas sensor. For example, the second calculation value SV2 is an estimated value corresponding to “R_{H2_2}” in Expression (3).

Here, in a case where there are measurement variations of the first gas sensor, the measurement variations are also reflected in the second calculation value SV2. FIG. 12 illustrates an example in which a measurement error for the second calculation value SV2 caused by the measurement variations of the first gas sensor is ±20%. A measurement error ME2 in FIG. 12 is obtained by visualizing the measurement error of ±20% for the second calculation value SV2, and the second calculation value SV2 can vary between an upper bound and a lower bound of the measurement error ME2.

The second calculation value SV2 is a value corresponding to a length up to the upper bound of the measurement error ME2 in a case where the measurement error of the first gas sensor reaches a maximum negative value (for example, the measurement error of −20%). In this case, the second calculation value SV2 is the smallest value, and the amount of the hydrogen gas is estimated to be small.

The second calculation value SV2 is a value corresponding to the length up to the lower bound of the measurement error ME2 in a case where the measurement error of the first gas sensor reaches a maximum positive value (for example, the measurement error of +20%). In this case, the second calculation value SV2 is the largest value, and the amount of the hydrogen gas is estimated to be large.

A third calculation value TV2 is calculated as the third calculation value corresponding to the odoriferous gas based on the 0-th calculation value ZV2 and the second calculation value SV2 (Step S21). For example, the third calculation value TV2 is an estimated value corresponding to “R_{H2S_2}” in Expression (3).

Here, the measurement error of ±20% for the second calculation value SV2 affects the third calculation value TV2. An error range ER2 in FIG. 12 is obtained by visualizing an error for the third calculation value TV2 that can occur due to the measurement error for the second calculation value SV2, and in a case where the measurement error for the second calculation value SV2 is ±20%, the third calculation value TV2 can vary between an upper bound and a lower bound of the error range ER2.

In a case where the measurement error for the second calculation value SV2 is −20%, the third calculation value TV2 is a value corresponding to a length up to the upper bound of the error range ER2. In this case, the third calculation value TV2 is the largest value, and the amount of the odoriferous gas is estimated to be large.

In a case where the measurement error for the second calculation value SV2 is +20%, the third calculation value TV2 is a value corresponding to a length up to the lower bound of the error range ER2. In this case, the third calculation value TV2 is the smallest value, and the amount of the odoriferous gas is estimated to be small.

Next, a problem that may occur in a case where the amount of the hydrogen gas is large and the amount of the odoriferous gas is small will be described with reference to FIG. 13.

A 0-th calculation value ZV3 is the 0-th calculation value corresponding to the hydrogen gas and the odoriferous gas obtained based on the detection result of the measurement performed by the second gas sensor. The 0-th calculation value ZV3 in FIG. 13 includes a first mixed value HV3 that is a value corresponding to the hydrogen gas and a second mixed value OV3 that is a value corresponding to the odoriferous gas.

For example, the first mixed value HV3 is an accurate value (correct value) corresponding to “R_{H2_2}” in Expression (3). Further, for example, the second mixed value OV3 is an accurate value (correct value) corresponding to “R_{H2S_2}” in Expression (3). In FIG. 13, for the sake of explanation, the breakdown of the first mixed value HV3 and the second mixed value OV3 in the 0-th calculation value ZV3 is illustrated, but the first mixed value HV3 and the second mixed value OV3 are actually estimated based on the third calculation value described below.

A second calculation value SV3 in FIG. 13 is calculated based on the first calculation value corresponding to the hydrogen gas obtained based on the detection result of the measurement performed by the first gas sensor. The second calculation value SV3 is the second calculation value corresponding to the hydrogen gas of the second gas sensor. For example, the second calculation value SV3 is an estimated value corresponding to “R_{H2_2}” in Expression (3).

Here, in a case where there are measurement variations of the first gas sensor, the measurement variations are also reflected in the second calculation value SV3. FIG. 13 illustrates an example in which a measurement error for the second calculation value SV3 caused by the measurement variations of the first gas sensor is ±20%. A measurement error ME3 in FIG. 13 is obtained by visualizing the measurement error of ±20% for the second calculation value SV3, and the second calculation value SV3 can vary between an upper bound and a lower bound of the measurement error ME3.

The second calculation value SV3 is a value corresponding to a length up to the upper bound of the measurement error ME3 in a case where the measurement error of the first gas sensor reaches a maximum negative value (for example, the measurement error of −20%). In this case, the second calculation value SV3 is the smallest value, and the amount of the hydrogen gas is estimated to be small.

The second calculation value SV3 is a value corresponding to the length up to the lower bound of the measurement error ME3 in a case where the measurement error of the first gas sensor reaches a maximum positive value (for example, the measurement error of +20%). In this case, the second calculation value SV3 is the largest value, and the amount of the hydrogen gas is estimated to be large. In the example of FIG. 13, the amount of the hydrogen gas is large and the amount of the odoriferous gas is small, and thus, in a case where the measurement error of the first gas sensor is a positive value, the second calculation value SV3 can become larger than the 0-th calculation value ZV3.

A third calculation value TV3 is calculated as the third calculation value corresponding to the odoriferous gas based on the 0-th calculation value ZV3 and the second calculation value SV3 (Step S22). For example, the third calculation value TV3 is an estimated value corresponding to “R_{H2S_2}” in Expression (3).

Here, the measurement error of ±20% for the second calculation value SV3 affects the third calculation value TV3. An error range ER3 in FIG. 13 is obtained by visualizing an error for the third calculation value TV3 that can occur due to the measurement error for the second calculation value SV3, and in a case where the measurement error for the second calculation value SV3 is ±20%, the third calculation value TV3 can vary between an upper bound and a lower bound of the error range ER3.

In a case where the measurement error for the second calculation value SV3 is −20%, the third calculation value TV3 is a value corresponding to a length up to the upper bound of the error range ER3. In this case, the third calculation value TV3 is the largest value, and the amount of the odoriferous gas is estimated to be large.

In a case where the measurement error for the second calculation value SV3 is +20%, the third calculation value TV3 is a value corresponding to a length up to the lower bound of the error range ER3. In the case of such a third calculation value TV3, the second calculation value SV3 can become larger than the 0-th calculation value ZV3, and the amount of the odoriferous gas may become 0 or less.

As described above, in a case where there are measurement variations of the first gas sensor, the measurement variations of the first gas sensor affects the estimated value of the amount of the odoriferous gas. For example, in the case illustrated in FIG. 13 described above, an influence of the measurement variations of the first gas sensor for the amount of the hydrogen gas is large, and thus, in some cases, the calculation value derived from the odoriferous gas can be 0 or less, and the score cannot be appropriately calculated. In such a case, it is difficult to appropriately execute processing based on the gas measurement. Therefore, the biological information measurement system 1 solves the above problem by any one of the following first processing, second processing, and third processing, and enables the processing based on the gas measurement to be appropriately executed.

### 1-9. First Processing (Plurality of Times of Measurement)

The biological information measurement system 1 executes the first processing using a plurality of pieces of information in order to suppress the influence of the measurement variations of the first gas sensor. Specifically, the biological information measurement system 1 calculates the second calculation value corresponding to the hydrogen gas of the second gas sensor based on the plurality of calculation values including the first calculation value corresponding to the hydrogen gas obtained based on the detection result of the first gas sensor.

As a result, the biological information measurement system 1 can suppress the influence of the measurement variations caused by the measurement performed a plurality of times. Examples of the configuration and measurement of the biological information measurement system 1 in a case where the measurement is performed a plurality of times as described above will be described below.

### 1-9-1. First Measurement Example

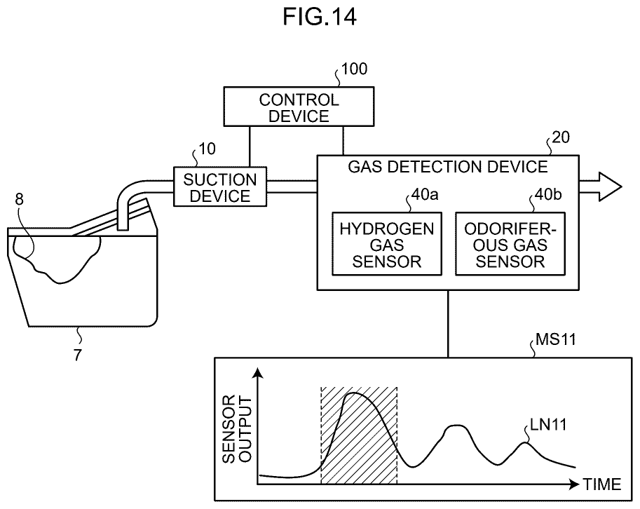

First, a first measurement example will be described with reference to FIG. 14. FIG. 14 is a diagram illustrating the first measurement example using the gas sensor. Specifically, FIG. 14 is a diagram illustrating an outline of the first measurement example which is processing using a plurality of pieces of data. In FIG. 14, only a part of the configuration of biological information measurement system 1 is illustrated in order to illustrate an image of the processing. In FIG. 14, the gas detection device 20 includes the gas sensor 40a that is the hydrogen gas sensor and the gas sensor 40b that is the odoriferous gas sensor. A description of matters similar to the contents described above will be omitted as appropriate.

Measurement MS11 in FIG. 14 corresponds to the measurement performed by the gas sensor 40a. For example, a line LN11 in the measurement MS11 indicates a sensor output that is the measurement value (voltage value) of the gas sensor 40a. A hatched portion in the measurement MS11 corresponds to one cycle of measurement processing, and indicates a change in the sensor output caused by the fecal gas emitted at the same time as a single defecation event, a single occurrence of flatulence, or the like. For example, the biological information measurement system 1 calculates the calculation value by using the maximum value (peak value) of one cycle of the measurement processing as the measurement value (voltage value).

In the first measurement example, the biological information measurement system 1 acquires a plurality of pieces of data for the fecal gas from a single defecation event or a single occurrence of flatulence by using the gas sensor 40a and averages the plurality of pieces of data. For example, the biological information measurement system 1 performs the measurement using the gas sensor 40a a plurality of times and averages a plurality of calculation values calculated by acquiring a plurality of measurement values. For example, the biological information measurement system 1 calculates the second calculation value by using a value obtained by averaging the corresponding calculation values of the plurality of times of measurement performed by the gas sensor 40a as a value of “R_{H2_1}” (also referred to as a “determined value”) in Expression (2).

As described above, the biological information measurement system 1 calculates the second calculation value that is the statistical value of the plurality of calculation values obtained by measuring the gas using the gas sensor 40a a plurality of times. In this case, the second calculation value is the statistical value of the plurality of calculation values obtained by measuring the gas using the gas sensor 40a a plurality of times. As a result, the biological information measurement system 1 can calculate (estimate) the amount of the hydrogen gas close to a true value by averaging the plurality of pieces of data and reducing the measurement variations. In the above example, a case where the average value is used as the determined value for the calculation of the second calculation value has been described as an example. However, the determined value is not limited to the average value as long as the determined value is determined based on the plurality of pieces of data, and the determined value used for the calculation of the second calculation value may be an arbitrary value such as a median value.

### 1-9-2. Second Measurement Example

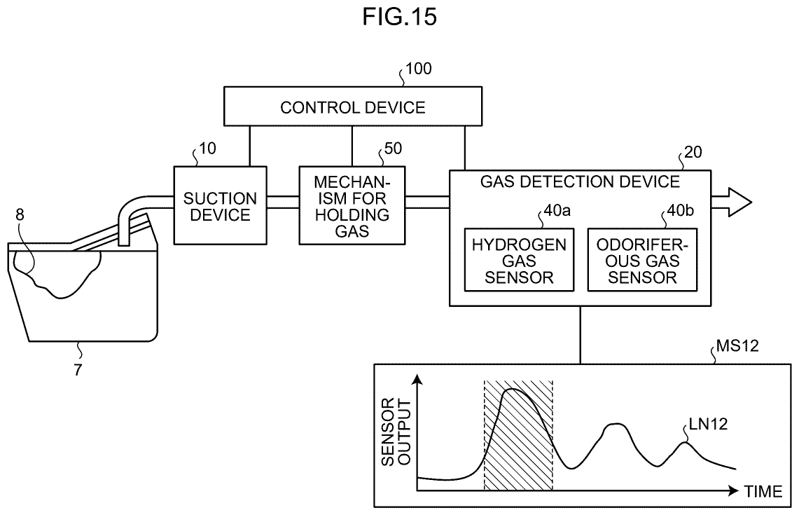

Next, a second measurement example will be described with reference to FIG. 15. FIG. 15 is a diagram illustrating the second measurement example using the gas sensor. Specifically, FIG. 15 is a diagram illustrating an outline of the second measurement example of the biological information measurement system 1 having a specific configuration for acquiring a plurality of pieces of data. A description of matters similar to the contents described above will be omitted as appropriate.

In FIG. 15, the biological information measurement system 1 includes sealing means 50 which is a mechanism for holding the gas. The sealing means 50 has a sealed space therein, and can hold the gas in the sealed space. In FIG. 15, the sealing means 50 is disposed between the gas detection device 20 and the suction device 10. That is, the sealing means 50 is disposed in the flow path between the gas detection device 20 and the suction device 10. The sealing means 50 holds the gas sucked by the suction device 10 in the sealed space. For example, the sealing means 50 includes a storage portion that stores the gas sucked by the suction device 10. The storage portion has a sealed space therein, and can store the gas in the sealed space.

In the second measurement example, the biological information measurement system 1 stores the fecal gas in the sealing means 50 in order to perform the measurement a plurality of times. In this case, the biological information measurement system 1 measures the held gas a plurality of times by using the mechanism for holding the gas, such as the sealing means 50. For example, in a case where a sensed output value of the gas sensor is equal to or larger than a predetermined value, the biological information measurement system 1 closes the closing valve (not illustrated). Then, the biological information measurement system 1 stops the suction device 10 and holds the gas in the storage portion. Then, the biological information measurement system 1 brings the held gas into contact with the gas sensor 40a.

Measurement MS12 in FIG. 15 corresponds to the measurement performed by the gas sensor 40a. For example, a line LN12 in the measurement MS12 indicates the sensor output that is the measurement value (voltage value) of the gas sensor 40a. A hatched portion in the measurement MS12 corresponds to one cycle of measurement processing, and indicates a change in the sensor output caused by the fecal gas emitted at the same time as a single defecation event, a single occurrence of flatulence, or the like. For example, the biological information measurement system 1 calculates the calculation value by using the maximum value (peak value) of one cycle of the measurement processing as the measurement value (voltage value).

In the second measurement example, the biological information measurement system 1 acquires a plurality of calculation values by a plurality of times of measurement performed by the gas sensor 40a that is the hydrogen gas sensor, after the fecal gas is held in the flow path (the sealing means 50 or the like), and averages the calculation values.

As described above, the biological information measurement system 1 calculates the second calculation value that is a statistical value of the plurality of calculation values obtained by measuring the gas in the sealed space using the gas sensor 40a a plurality of times. The second calculation value is the statistical value of the plurality of calculation values obtained by measuring the gas in the sealed space using the gas sensor 40a a plurality of times. Specifically, the biological information measurement system 1 calculates the second calculation value that is the statistical value of the plurality of calculation values obtained by measuring the gas in the storage portion a plurality of times. The second calculation value is the statistical value of the plurality of calculation values obtained by measuring the gas in the storage portion a plurality of times.

As a result, the biological information measurement system 1 can measure the fecal gas many times by using the hydrogen gas sensor by holding the fecal gas in the flow path, and can reduce an influence of the measurement variations of the sensor by averaging a plurality of signals. Processing after calculating the second calculation value is similar to that in the first measurement example, and thus a detailed description thereof will be omitted.

### 1-9-3. Third Measurement Example

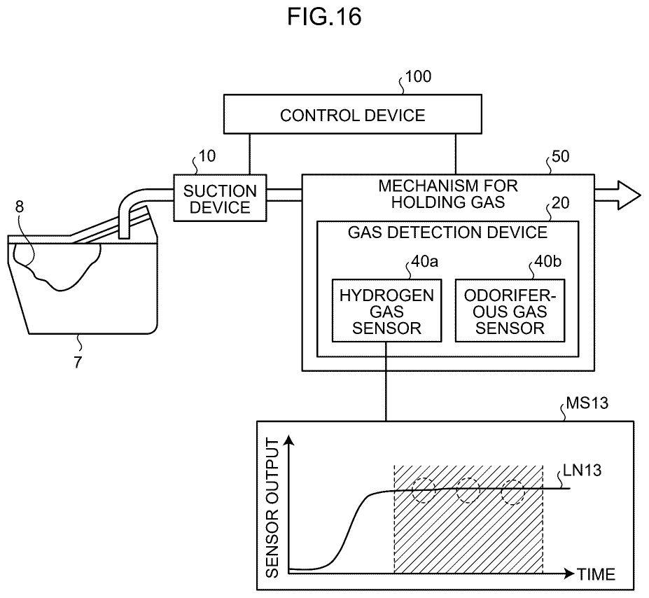

Next, a third measurement example will be described with reference to FIG. 16. FIG. 16 is a diagram illustrating the third measurement example using the gas sensor. Specifically, FIG. 16 is a diagram illustrating an outline of the third measurement example of the biological information measurement system 1 in which the gas detection device 20 is disposed in the sealing means 50. A description of matters similar to the contents described above will be omitted as appropriate.

In FIG. 16, the biological information measurement system 1 includes the sealing means 50. In FIG. 16, the sealing means 50 accommodates the gas detection device 20. In the biological information measurement system 1 of the third measurement example, the gas detection device 20 is disposed in the sealing means 50. The sealing means 50 has a flow path switchable to a closed flow path by opening/closing means (for example, the switching valve) described below.

In the third measurement example, the biological information measurement system 1 measures the gas held in the sealing means 50 a plurality of times by using the gas detection device 20 in the sealing means 50. In this case, the biological information measurement system 1 acquires data a plurality of times in a state in which the gas covers the entire sensor surface and a sensor output is stable.

Measurement MS13 in FIG. 16 corresponds to the measurement performed by the gas sensor 40a. For example, a line LN13 in the measurement MS13 indicates the sensor output that is the measurement value (voltage value) of the gas sensor 40a. The hatched portion in the measurement MS13 corresponds to a part of a section in which the sensor output (power value) reaches a peak (maximum) due to the gas held in the sealing means 50. For example, the biological information measurement system 1 executes the measurement processing a plurality of times (three times as indicated by dotted circles in FIG. 16) in the section in which the sensor output (power value) reaches the peak (maximum), and calculates the calculation value corresponding to each step of measurement processing.

In the third measurement example, the biological information measurement system 1 stores the fecal gas in the sealing means 50 to perform the measurement a plurality of times. The biological information measurement system 1 holds the fecal gas in the sealing means 50 accommodating the gas detection device 20, acquires a plurality of calculation values by a plurality of times of measurement performed by the gas sensor 40a that is the hydrogen gas sensor, and averages the calculation values.

As described above, the biological information measurement system 1 calculates the second calculation value that is a statistical value of a plurality of calculation values obtained by measuring the gas in the sealing means 50 using the gas sensor 40a a plurality of times. The second calculation value is the statistical value of the plurality of calculation values obtained by measuring the gas in the sealing means 50 using the gas sensor 40a a plurality of times.

As a result, the biological information measurement system 1 can acquire a signal in a state in which the sensor output is stable by continuously bringing the fecal gas into contact with the hydrogen gas sensor, and can further reduce the influence of the measurement variations of the gas sensor by averaging. Processing after calculating the second calculation value is similar to that in the first measurement example, and thus a detailed description thereof will be omitted.

### 1-9-4. Fourth Measurement Example

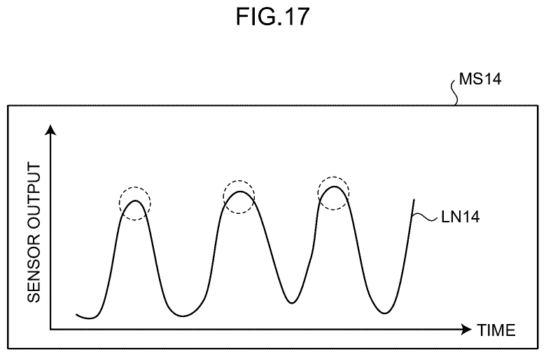

Next, a fourth measurement example will be described with reference to FIG. 17. FIG. 17 is a diagram illustrating the fourth measurement example using the gas sensor. FIG. 17 is a diagram illustrating an outline of the fourth measurement example of the biological information measurement system 1 with a configuration similar to that of the second measurement example illustrated in FIG. 15. Specifically, FIG. 17 is a diagram illustrating the fourth measurement example in which the gas held in the flow path between the gas detection device 20 and the suction device 10 by the sealing means 50 is measured. A description of matters similar to the contents described above will be omitted as appropriate.

Since the configuration of the biological information measurement system 1 in the fourth measurement example is similar to the configuration of the biological information measurement system 1 in the second measurement example, the illustration and detailed description thereof will be omitted.

Measurement MS14 in FIG. 17 corresponds to the measurement performed by the gas sensor 40a. For example, a line LN14 in MS14 indicates the sensor output that is the measurement value (voltage value) of the gas sensor 40a. For example, the biological information measurement system 1 executes the measurement processing (three times in FIG. 17) at each time point when the sensor output (power value) reaches a peak (maximum) as indicated by a dotted circle in FIG. 17, and calculates the calculation value corresponding to each step of measurement processing. For example, the biological information measurement system 1 repeatedly brings the gas into contact with the sensor to acquire a plurality of pieces of data corresponding to the peak value of the sensor output.

In the fourth measurement example, the biological information measurement system 1 stores the fecal gas in the flow path between the gas detection device 20 and the suction device 10 by the sealing means 50 in order to measure the fecal gas a plurality of times. The biological information measurement system 1 holds the fecal gas in the flow path between the gas detection device 20 and the suction device 10, acquires a plurality of calculation values by a plurality of times of measurement performed by the gas sensor 40a that is the hydrogen gas sensor, and averages the calculation values. That is, the biological information measurement system 1 holds the fecal gas at a place different from the inside of the gas detection device 20, then repeatedly brings the gas into contact with the gas sensor 40a that is the hydrogen gas sensor, acquires a plurality of calculation values by a plurality of times of measurement performed by the gas sensor 40a, and averages the plurality of calculation values.

As described above, the biological information measurement system 1 calculates the second calculation value that is the statistical value of the plurality of calculation values obtained by measuring the gas in the flow path a plurality of times. The second calculation value is the statistical value of the plurality of calculation values obtained by measuring the gas in the flow path a plurality of times.

As a result, the biological information measurement system 1 can reduce an influence of the measurement error by repeatedly measuring the held fecal gas using the hydrogen gas sensor and averaging a plurality of first calculation values acquired. Processing after calculating the second calculation value is similar to that in the first measurement example, and thus a detailed description thereof will be omitted.

### 1-9-5. Fifth Measurement Example

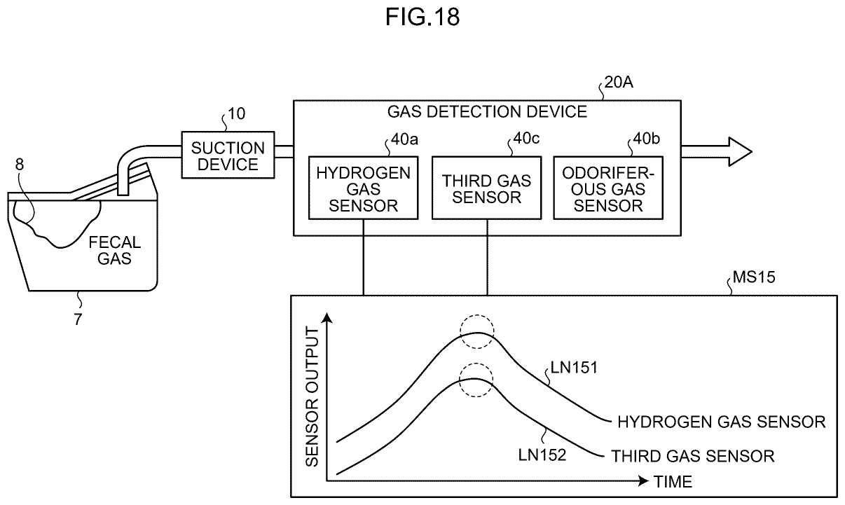

Next, a fifth measurement example will be described with reference to FIG. 18. FIG. 18 is a diagram illustrating the fifth measurement example using the gas sensor. Specifically, FIG. 18 is a diagram illustrating an outline of the fifth measurement example of the biological information measurement system 1 in which the gas detection device 20 (referred to as a “gas detection device 20A”) including the gas sensor 40c that is the third gas sensor is disposed. A description of matters similar to the contents described above will be omitted as appropriate.

In FIG. 18, the biological information measurement system 1 includes the gas detection device 20A including the gas sensor 40c. For example, the gas sensor 40c that is the third gas sensor is a gas sensor having higher sensitivity to the hydrogen gas and lower sensitivity to the odoriferous gas than the gas sensor 40b. In the fifth measurement example, the gas sensor 40c may be the hydrogen gas sensor.

In the fifth measurement example, the biological information measurement system 1 acquires values (for example, calculation values) indicating the amount and concentration of the hydrogen gas from the gas sensor 40a that is the hydrogen gas sensor and the gas sensor 40c that is the third gas sensor, and averages the values.

The biological information measurement system 1 calculates a calculation value (also referred to as a “fourth calculation value”) corresponding to the hydrogen gas based on the detection result of the gas sensor 40c. For example, the biological information measurement system 1 calculates the fourth calculation value corresponding to the hydrogen gas of the gas sensor 40c from the measurement value (voltage value) of the gas sensor 40c using Expression (1). The biological information measurement system 1 calculates (estimates) the amount of the hydrogen gas based on the measurement performed by the gas sensor 40c by applying the fourth calculation value to the regression equation.

Measurement MS15 in FIG. 18 corresponds to measurement performed by the gas sensor 40a and the gas sensor 40c. For example, a line LN151 in the measurement MS15 indicates the sensor output that is the measurement value (voltage value) of the gas sensor 40a. For example, a line LN152 in the measurement MS15 indicates a sensor output that is a measurement value (voltage value) of the gas sensor 40c.

For example, the biological information measurement system 1 executes the measurement processing at a time point when the sensor output (power value) of the gas sensor 40a reaches a peak (maximum) as indicated by a dotted circle on the line LN151 in FIG. 18, and calculates the first calculation value corresponding to the measurement processing. In addition, for example, the biological information measurement system 1 executes the measurement processing at a time point when the sensor output (power value) of the gas sensor 40c reaches a peak (maximum) as indicated by a dotted circle on the line LN152 in FIG. 18, and calculates the fourth calculation value corresponding to the measurement processing. For example, the biological information measurement system 1 acquires data corresponding to the peak values of the hydrogen gas sensor and the third gas sensor.

In the fifth measurement example, the biological information measurement system 1, the biological information measurement system 1 calculates the second calculation value by using the first calculation value based on the measurement performed by the gas sensor 40a and the fourth calculation value based on the measurement performed by the gas sensor 40c.

As described above, the biological information measurement system 1 calculates the second calculation value that is a statistical value obtained using a plurality of calculation values obtained by measurement performed by the plurality of gas sensors 40 including the gas sensor 40a and the gas sensor 40c. The second calculation value is obtained from the statistical value of the plurality of calculation values obtained by measurement performed by the plurality of gas sensors 40.

As a result, the biological information measurement system 1 calculates values (for example, the calculation values) indicating the amounts of the hydrogen gas from the plurality of gas sensors and averages the values to reduce individual variations and calculate a more correct amount of the hydrogen gas. Processing after calculating the second calculation value is similar to that in the first measurement example, and thus a detailed description thereof will be omitted.

### 1-9-6. Sixth Measurement Example

Next, a sixth measurement example will be described. The biological information measurement system 1 of the sixth measurement example is different from the biological information measurement system 1 of the fifth measurement example in that the gas detection device 20A includes a gas sensor of a type different from the hydrogen gas sensor as the gas sensor 40c that is the third gas sensor. A description of matters similar to the contents described above will be omitted as appropriate.

In the sixth measurement example, the biological information measurement system 1 includes the gas detection device 20A including the gas sensor 40c that is the methane gas sensor different from the gas sensor 40a that is the hydrogen gas sensor. For example, the gas sensor 40c that is the methane gas sensor is a gas sensor that easily reacts with hydrogen and methane and hardly reacts with the odoriferous gas. For example, the gas sensor 40c that is the methane gas sensor is mounted on the gas detection device 20 in order to measure the methane gas in the fecal gas.

Here, the number of people having methanogen is small, and a percentage of people whose the fecal gas contains the methane gas is low. For example, in the case of a person who does not emit the methane gas, the methane gas sensor can calculate the amount of the hydrogen gas.

Therefore, in the sixth measurement example, the biological information measurement system 1 uses the gas sensor 40c as a methane gas measurement sensor in a case where a person who emits the methane gas is a measurement subject. In a case where it is determined that the methane gas is contained in the fecal gas of the user, the biological information measurement system 1 uses the gas sensor 40c as a sensor for detecting the methane gas.

On the other hand, in a case where a person who does not emit the methane gas is the measurement target, the biological information measurement system 1 uses the gas sensor 40c as a hydrogen gas measurement sensor. In a case where it is determined that the methane gas is not contained in the fecal gas of the user, the biological information measurement system 1 uses the gas sensor 40c as a sensor for detecting the hydrogen gas.

As described above, in a case where a gas sensor intended to measure other components is not used for the original purpose, the biological information measurement system 1 can further improve the measurement accuracy of the hydrogen gas sensor by repurposing the sensor as a sensor for measuring the hydrogen gas.

For example, when calculating the fourth calculation value of the gas sensor 40c that is that methane gas sensor, the biological information measurement system 1 may calculate the fourth calculation value by using an expression for the methane gas sensor instead of Expressions (3), (5), and (6) for the odoriferous gas. In this case, the calculation of the fourth calculation value is similar to the calculation of the second calculation value of the odoriferous gas except that the expression to be used is different, and thus a detailed description thereof will be omitted.

### 1-9-7. Seventh Measurement Example

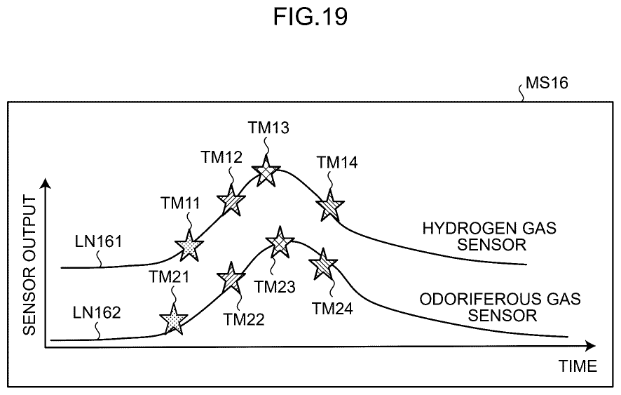

Next, a seventh measurement example will be described with reference to FIG. 19. FIG. 19 is a diagram illustrating the seventh measurement example using the gas sensor. FIG. 19 is a diagram illustrating an outline of the seventh measurement example of the biological information measurement system 1 with a configuration similar to that of any of the first to sixth measurement examples described above. For example, the seventh measurement example is performed by the biological information measurement system 1 configured as in the second measurement example illustrated in FIG. 15. A description of matters similar to the contents described above will be omitted as appropriate.

Measurement MS16 in FIG. 19 corresponds to the measurement performed by the gas sensor 40a and the gas sensor 40b. For example, a line LN161 in the measurement MS16 indicates the sensor output that is the measurement value (voltage value) of the gas sensor 40a. For example, a line LN162 in the measurement MS16 indicates the sensor output that is the measurement value (voltage value) of the gas sensor 40b. A responsiveness of each gas sensor 40 is determined by any means. For example, the responsiveness of each gas sensor 40 is set such that a peak value is 100% and a value before defecation is 0%.

For example, a measurement value TM11 on the line LN161 in FIG. 19 indicates a measurement value at a time point when the responsiveness of the gas sensor 40a reaches a first level (for example, 30% of the peak value). A measurement value TM12 on the line LN161 in FIG. 19 indicates a measurement value at a time point when the responsiveness of the gas sensor 40a reaches a second level (for example, 50% of the peak value). A measurement value TM13 on the line LN161 in FIG. 19 indicates a measurement value at a time point when the responsiveness of the gas sensor 40a reaches a third level (for example, the peak value).

For example, a measurement value TM21 on the line LN162 in FIG. 19 indicates a measurement value at a time point when the responsiveness of the gas sensor 40b reaches the first level (for example, 30% of the peak value). A measurement value TM22 on the line LN162 in FIG. 19 indicates a measurement value at a time point when the responsiveness of the gas sensor 40b reaches the second level (for example, 50% of the peak value). A measurement value TM23 on the line LN162 in FIG. 19 indicates a measurement value at a time point when the responsiveness of the gas sensor 40b reaches the third level (for example, the peak value).

In FIG. 19, the biological information measurement system 1 uses, as a set, values of the plurality of gas sensors 40 at a timing when the levels of responsiveness to the fecal gas are the same. For example, the biological information measurement system 1 uses, as one set, the measurement value TM11 of the gas sensor 40a and the measurement value TM21 of the gas sensor 40b at a timing when both the levels of responsiveness of the gas sensor 40a and the gas sensor 40b are the first level.

In this case, the biological information measurement system 1 calculates a first calculation value (referred to as a “first calculation value FV71”) and a third calculation value (referred to as a “third calculation value TV71”) based on the measurement value TM11 and the measurement value TM21. Then, the biological information measurement system 1 calculates the amount of the hydrogen gas (referred to as a “hydrogen gas amount VL11”) and the amount of the odoriferous gas (referred to as an “odoriferous gas amount VL21”) based on the calculated first calculation value FV71 and third calculation value TV71. Then, the biological information measurement system 1 calculates a primary score (referred to as a “primary score TS1”) by obtaining a ratio between the hydrogen gas amount VL11 and the odoriferous gas amount VL21 that are calculated.

Further, the biological information measurement system 1 uses, as one set, the measurement value TM12 of the gas sensor 40a and the measurement value TM22 of the gas sensor 40b at a timing when both the levels of responsiveness of the gas sensor 40a and the gas sensor 40b are the second level. In this case, the biological information measurement system 1 calculates the first calculation value FV72 and the third calculation value TV72 based on the measurement value TM12 and the measurement value TM22, and calculates the amount of the hydrogen gas (referred to as a “hydrogen gas amount VL12”) and the amount of the odoriferous gas (referred to as an “odoriferous gas amount VL22”) based on the calculated first calculation value FV72 and third calculation value TV72. Then, the biological information measurement system 1 calculates a primary score (referred to as a “primary score TS2”) by obtaining a ratio between the hydrogen gas amount VL12 and the odoriferous gas amount VL22 that are calculated.

Further, the biological information measurement system 1 uses, as one set, the measurement value TM13 of the gas sensor 40a and the measurement value TM23 of the gas sensor 40b at a timing when both the levels of responsiveness of the gas sensor 40a and the gas sensor 40b are the third level (peak value). In this case, the biological information measurement system 1 calculates a first calculation value FV73 and a third calculation value TV73 based on the measurement value TM13 and the measurement value TM23, and calculates the amount of the hydrogen gas (referred to as a “hydrogen gas amount VL13”) and the amount of the odoriferous gas (referred to as an “odoriferous gas amount VL23”) based on the calculated first calculation value FV72 and third calculation value TV72. Then, the biological information measurement system 1 calculates a primary score (referred to as a “primary score TS3”) by obtaining a ratio between the hydrogen gas amount VL13 and the odoriferous gas amount VL23 that are calculated.

As a result, the biological information measurement system 1 acquires a plurality of ratios of values based on the measurement performed by the hydrogen gas sensor and the measurement performed by the odoriferous gas sensor. Then, the biological information measurement system 1 calculates a score by averaging the plurality of acquired ratios (for example, the primary score). In FIG. 19, the biological information measurement system 1 calculates an average value of the primary score TS1, the primary score TS2, and the primary score TS3 as the score.

As described above, in the seventh measurement example, the biological information measurement system 1 acquires a plurality of values based on the measurement performed by the hydrogen gas sensor and the odoriferous gas sensor at the same timing, and calculates ratios thereof, and averages the ratios. The biological information measurement system 1 estimates the health condition of the user or the information regarding the health condition by using the score calculated based on the first calculation value corresponding to the hydrogen gas obtained based on the detection result of the first gas sensor and the third calculation value. The biological information measurement system 1 calculates a score based on a ratio between a value (for example, the amount of the hydrogen gas) based on the first calculation value and a value (the amount of the odoriferous gas) based on the third calculation value corresponding to detection at a common timing in the defecation of the user.

As described above, the biological information measurement system 1 acquires a value derived from hydrogen and a value derived from the odoriferous gas at the same timing in one defecation act. The biological information measurement system 1 averages ratios between values (for example, the amounts of the hydrogen gas) based on the first calculation value and values (the amounts of the odoriferous gas) based on the third calculation value in one set, and calculates the ratio between the value based on the first calculation value and the value based on the third calculation value. As described above, the biological information measurement system 1 calculates, as the score, a statistical value of the ratios of the values based on the plurality of calculation values.

Variations in the score (for example, the ratio of the amounts) are greatly affected by the variations caused by the hydrogen gas sensor. Therefore, the biological information measurement system 1 can reduce the variations derived from the hydrogen gas sensor as a result by averaging the scores (for example, the ratios of the amounts).

### 1-9-8. Configuration and Control Example

Hereinafter, an example of a configuration and control for the above-described measurement examples will be described. The configuration and control of the biological information measurement system 1 described below are merely examples, and any configuration and control can be adopted in the biological information measurement system 1 as long as the above-described measurement can be performed.

### 1-9-8-1. Configuration and Control Corresponding to Third Measurement Example

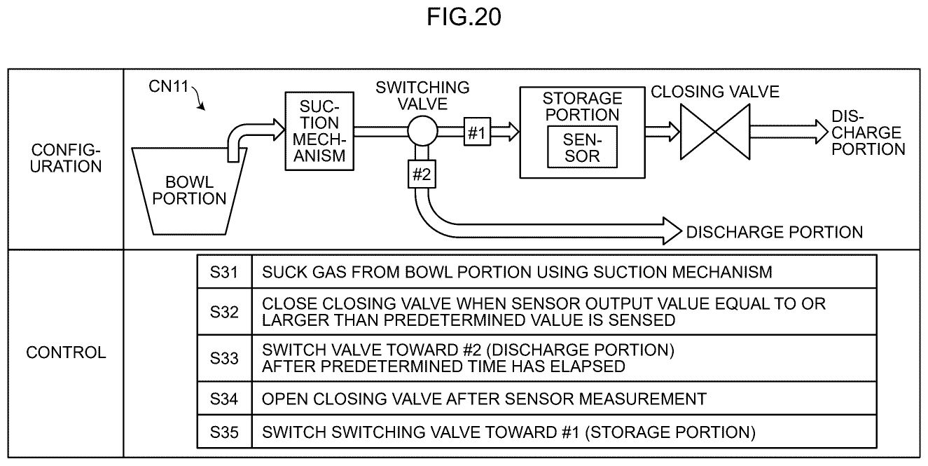

First, a configuration and control corresponding to the third measurement example will be described with reference to FIG. 20. FIG. 20 is a diagram illustrating an example of the configuration and control corresponding to the third measurement example. A description of matters similar to the contents described above will be omitted as appropriate.

A device configuration CN11 illustrated in FIG. 20 is an example of a device configuration of the biological information measurement system 1 that performs the third measurement example. FIG. 20 illustrates a concept of the configuration and control, and the device configuration CN11 is only a part of the configuration of the biological information measurement system 1 that performs the third measurement example. For example, a suction mechanism of the device configuration CN11 corresponds to the suction device 10, a discharge portion corresponds to the duct 12, and a sensor corresponds to the gas sensor 40. Further, for example, a switching valve, a flow path #1, a flow path #2, a storage portion, and a closing valve are included in the sealing means 50.

In FIG. 20, the biological information measurement system 1 executes processing in Steps S31 to S35. Before Step S31, the switching valve is set such that the gas in the bowl portion flows toward the flow path #1. The biological information measurement system 1 sucks the gas from the bowl portion with the suction mechanism (Step S31). When a sensor output value (for example, the voltage value) equal to or larger than a predetermined value is sensed, the biological information measurement system 1 closes the closing valve (Step S32).

After the lapse of a predetermined time, the biological information measurement system 1 switches the switching valve toward the flow path #2 (the gas directly flows to the discharge portion) (Step S33). After the sensor measurement, the biological information measurement system 1 opens the closing valve (Step S34). The biological information measurement system 1 switches the switching valve toward the flow path #1 (the gas flows to the storage portion) (Step S35).

As described above, in FIG. 20, the biological information measurement system 1 performs the measurement by using the sensor in a state in which the closing valve is closed and the gas is stored.

### 1-9-8-2. Configuration and Control Corresponding to Fourth Measurement Example

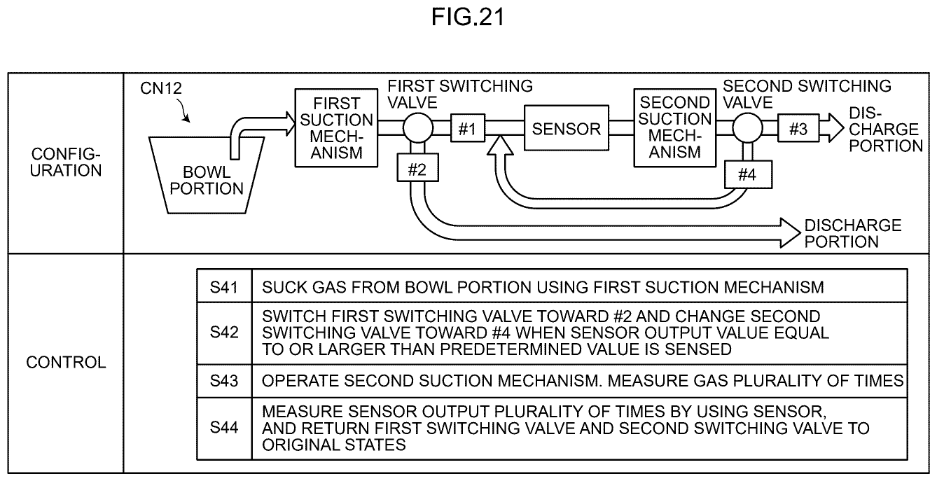

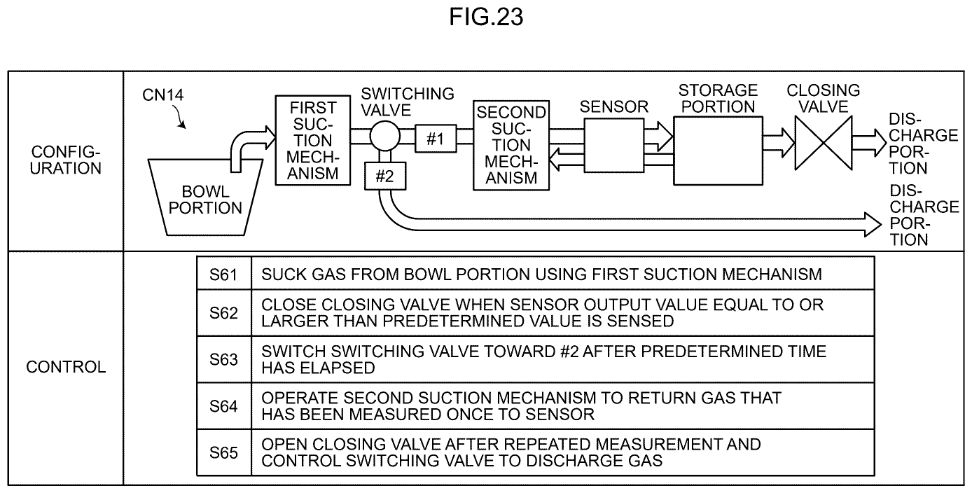

Next, a configuration and control corresponding to the fourth measurement example will be described with reference to FIGS. 21 to 23. FIGS. 21 and 23 are diagrams illustrating an example of the configuration and control corresponding to the fourth measurement example. A description of matters similar to the contents described above will be omitted as appropriate.

A configuration and control in a case where the gas is circulated and measured a plurality of times will be described with reference to FIG. 21. A device configuration CN12 illustrated in FIG. 21 is an example of a device configuration of the biological information measurement system 1 that performs the fourth measurement example. FIG. 21 illustrates a concept of the configuration and control, and the device configuration CN12 is only a part of the configuration of the biological information measurement system 1 that performs the fourth measurement example. For example, a first suction mechanism of the device configuration CN12 corresponds to the suction device 10, a discharge portion corresponds to the duct 12, and a sensor corresponds to the gas sensor 40. Further, for example, a first switching valve, a flow path #1, a flow path #2, a second suction mechanism, a second switching valve, a flow path #3, and a flow path #4 are included in the sealing means 50. For example, the second suction mechanism is a device having a function similar to that of the suction device 10, and can perform suction in a desired direction.

In FIG. 21, the biological information measurement system 1 executes processing in Steps S41 to S44. Before Step S41, the first switching valve is set such that the gas in the bowl portion flows toward the flow path #1, and the second switching valve is set such that the gas in the bowl portion flows toward the flow path #3. The biological information measurement system 1 sucks the gas from the bowl portion with the first suction mechanism (Step S41). When the sensor output value (for example, the voltage value) equal to or larger than a predetermined value is sensed, the biological information measurement system 1 switches the first switching valve toward the flow path #2 (the gas directly flows to the discharge portion), and switches the second switching valve toward the flow path #4 (the gas is circulated in the sealing means 50) (Step S42).

The biological information measurement system 1 operates the second suction mechanism and measures the gas a plurality of times (Step S43). After measuring the sensor output a plurality of times by the sensor, the biological information measurement system 1 returns the first switching valve and the second switching valve to the original states (Step S44). In FIG. 21, the biological information measurement system 1 switches the first switching valve such that the gas in the bowl portion flows toward the flow path #1, and switches the second switching valve such that the gas flows toward the flow path #3.

As described above, in FIG. 21, the biological information measurement system 1 circulates the same gas in the flow path and repeatedly measures the gas with the sensor.

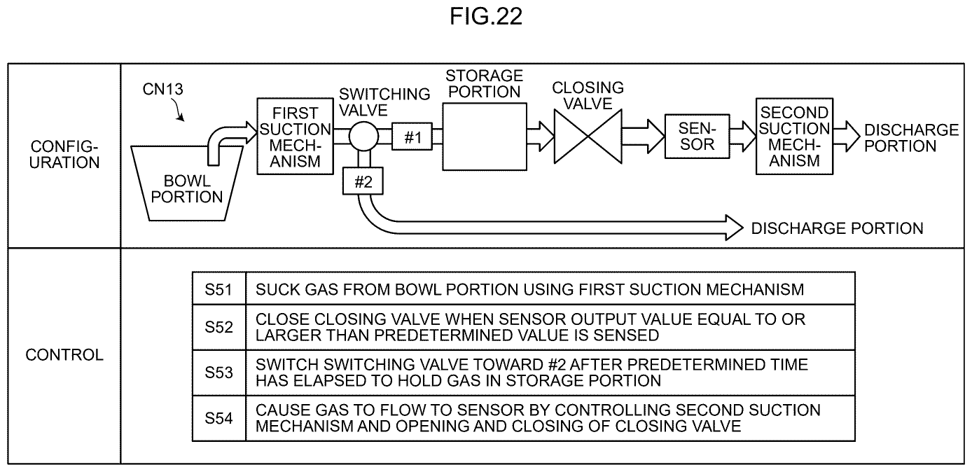

A configuration and control in a case where the stored gas is dispensed and measured a plurality of times will be described with reference to FIG. 22. A device configuration CN13 illustrated in FIG. 22 is an example of a device configuration of the biological information measurement system 1 that performs the fourth measurement example. FIG. 22 illustrates a concept of the configuration and control, and the device configuration CN13 is only a part of the configuration of the biological information measurement system 1 that performs the fourth measurement example. For example, a first suction mechanism of the device configuration CN13 corresponds to the suction device 10, a discharge portion corresponds to the duct 12, and a sensor corresponds to the gas sensor 40. Further, for example, a switching valve, a flow path #1, a flow path #2, a storage portion, a closing valve, and a second suction mechanism are included in the sealing means 50.

In FIG. 22, the biological information measurement system 1 executes processing in Steps S51 to S54. Before Step S51, the switching valve is set such that the gas in the bowl portion flows toward the flow path #1. The biological information measurement system 1 sucks the gas from the bowl portion with the first suction mechanism (Step S51). When a sensor output value (for example, the voltage value) equal to or larger than a predetermined value is sensed, the biological information measurement system 1 closes the closing valve (Step S52).

After the lapse of a predetermined time, the biological information measurement system 1 switches the switching valve toward the flow path #2 (the gas directly flows to the discharge portion) and holds the gas in the storage portion (Step S53). The biological information measurement system 1 causes the gas to flow to the sensor by controlling the second suction mechanism and opening and closing of the closing valve (Step S54). In this case, for example, while the gas is measured by the sensor, the biological information measurement system 1 opens the closing valve and operates the second suction mechanism to discharge a part of the gas in the storage portion from the discharge portion via the sensor. In addition, while the measurement of the gas by the sensor is stopped, the biological information measurement system 1 closes the closing valve, stops the second suction mechanism, and stops the discharge of the gas in the storage portion through the discharge portion, for example.

As described above, in FIG. 22, the biological information measurement system 1 holds the gas in the storage portion, and periodically causes a predetermined amount of gas to flow to the sensor.

A configuration and control in a case where the measurement is performed a plurality of times by returning the gas that has been measured once will be described with reference to FIG. 23. A device configuration CN14 illustrated in FIG. 23 is an example of a device configuration of the biological information measurement system 1 that performs the fourth measurement example. FIG. 23 illustrates a concept of the configuration and control, and the device configuration CN14 is only a part of the configuration of the biological information measurement system 1 that performs the fourth measurement example. For example, a first suction mechanism of the device configuration CN14 corresponds to the suction device 10, a discharge portion corresponds to the duct 12, and a sensor corresponds to the gas sensor 40. Further, for example, a switching valve, a flow path #1, a flow path #2, a storage portion, a closing valve, and a second suction mechanism are included in the sealing means 50.

In FIG. 23, the biological information measurement system 1 executes processing in Steps S61 to S65. Before Step S61, the switching valve is set such that the gas in the bowl portion flows toward the flow path #1. The biological information measurement system 1 sucks the gas from the bowl portion with the first suction mechanism (Step S61). When a sensor output value (for example, the voltage value) equal to or larger than a predetermined value is sensed, the biological information measurement system 1 closes the closing valve (Step S62).

After the lapse of a predetermined time, the biological information measurement system 1 switches the switching valve toward the flow path #2 (the gas directly flows to the discharge portion) (Step S63). The biological information measurement system 1 operates the second suction mechanism to return the gas that has been measured once to the sensor (Step S64). A flow path between the second suction mechanism and the storage portion may be common, or a flow path from the second suction mechanism to the storage portion and a flow path from the storage portion to the second suction mechanism may be separately provided. After the repeated measurement, the biological information measurement system 1 opens the closing valve and controls the switching valve to discharge the gas (Step S65).

As described above, in FIG. 23, the biological information measurement system 1 holds the gas in the storage portion, and returns the gas measured using the second suction mechanism to the sensor.

### 1-10. Second Processing (Correction)

The biological information measurement system 1 may suppress the influence of the measurement variations by an arbitrary method other than the first processing described above. For example, in order to suppress the influence of the measurement variations of the gas sensor, the biological information measurement system 1 executes the second processing of performing value correction in a case where a value or the like based on the measurement satisfies a predetermined condition. In the second processing, the biological information measurement system 1 secondarily performs the correction in a case where a predetermined condition that there is a possibility that the odoriferous gas has not been correctly estimated is satisfied. Specifically, in a case where there is a possibility that the odoriferous gas has not been correctly estimated, the biological information measurement system 1 performs the correction on at least one of the 0-th calculation value, the second calculation value, or the third calculation value described above.

As a result, the biological information measurement system 1 can suppress the influence of the measurement variations by the correction. This matter will be described below. A description of matters similar to the contents described in the first processing and the like will be omitted as appropriate.

### 1-10-1. First Correction

First, an example in which the biological information measurement system 1 corrects the 0-th calculation value or the second calculation value will be described as first correction.

For example, in a case where the predetermined condition is satisfied, the biological information measurement system 1 performs the first correction. The biological information measurement system 1 performs the first correction in a case where the first calculation value or the second calculation value is larger than the first threshold or in a case where the third calculation value is smaller than the second threshold smaller than the first threshold. In this case, the biological information measurement system 1 performs the first correction in a case where at least one of correction conditions including a first condition that the first calculation value or the second calculation value is larger than the first threshold and a second condition that the third calculation value is smaller than the second threshold smaller than the first threshold is satisfied. The first threshold and the second threshold are set to arbitrary values according to the gas sensor 40 or the like. For example, the first threshold and the second threshold may be set for each biological information measurement system 1 (that is, for each device) into which the gas sensor 40 is introduced, or may be values set in common.

For example, in a case where at least one of the correction conditions is satisfied, the biological information measurement system 1 performs correction of decreasing the second calculation value. When calculating the second calculation value from the first calculation value, the biological information measurement system 1 performs the first correction by multiplying the second calculation value by a correction coefficient smaller than 1. In this case, the biological information measurement system 1 may apply any one of a correction pattern #1 and a correction pattern #2.

In a case where the correction pattern #1 is applied, when at least one of the correction conditions is satisfied, the biological information measurement system 1 corrects the first calculation value by multiplying the first calculation value by a correction coefficient (for example, a value smaller than 1). In this case, the biological information measurement system 1 calculates the second calculation value by using the calculated corrected first calculation value.

In a case where the correction pattern #2 is applied, when at least one of the correction conditions is satisfied, the biological information measurement system 1 corrects the second calculation value by multiplying the second calculation value by a correction coefficient (for example, a value smaller than 1). In this case, the biological information measurement system 1 calculates the third calculation value by using the calculated corrected second calculation value.

The above-described correction is merely an example, and the correction performed by the biological information measurement system 1 is not limited to the correction of decreasing the second calculation value. The biological information measurement system 1 may perform correction of increasing the 0-th calculation value. Further, the biological information measurement system 1 may perform the first correction in a case where the second calculation value is larger than the 0-th calculation value. In this case, a third condition that the second calculation value is larger than the 0-th calculation value may be included in the correction condition.

As described above, in the first correction, the correction is performed using another value used for calculating the third calculation value indicating the amount of the odoriferous gas as a correction target. As a result, the biological information measurement system 1 can appropriately correct the third calculation value calculated based on the another value and appropriately calculate (estimate) the amount of the odoriferous gas. Therefore, the biological information measurement system 1 can appropriately execute processing based on the gas measurement.

### 1-10-2. Second Correction

The biological information measurement system 1 may perform not only the first correction but also second correction of correcting the third calculation value. In this case, the biological information measurement system 1 has the correction value set in advance as the value corresponding to the odoriferous gas, and replaces the third calculation value with the correction value as the correction in a case where the third calculation value is smaller than the third threshold.

The third threshold is set to an arbitrary value according to the gas sensor 40 or the like. For example, the third threshold may be set for each biological information measurement system 1 (that is, for each device) into which the gas sensor 40 is introduced, or may be a value set in common.

The correction value can be set to an arbitrary value. For example, the correction value may be a lower limit detection value of the gas sensor, or may be a minimum value of a calculation value based on data with reference to data of an amount of a gas emitted from a person.

As described above, in the second correction, the correction is performed using the third calculation value itself indicating the amount of the odoriferous gas as the correction target. As a result, the biological information measurement system 1 can appropriately correct the third calculation value and appropriately calculate (estimate) the amount of the odoriferous gas. Therefore, the biological information measurement system 1 can appropriately execute processing based on the gas measurement.

### 1-11. Third Processing (Change of Information)

The biological information measurement system 1 may suppress the influence of the measurement variations by an arbitrary method other than the first processing and the second processing described above. For example, the biological information measurement system 1 executes the third processing of changing information to be output in order to suppress the influence of the measurement variations of the gas sensor. In the third processing, in a case where a condition (also referred to as a “change condition”) that there is a possibility that a ratio between the health-related gas and the odoriferous gas is incorrect is satisfied, the biological information measurement system 1 changes the information and outputs and displays the changed information. Specifically, in a case where at least one of the first calculation value, the second calculation value, and the third calculation value satisfies the change condition, the biological information measurement system 1 performs control to change the first information which is the health condition of the user or the information regarding the health condition of the user output by output means.

As a result, the biological information measurement system 1 can suppress the influence of the measurement variations by changing information to be provided to the user by changing the information. This matter will be described below. A description of points similar to the contents described in the first processing, the second processing, and the like will be omitted as appropriate.

### 1-11-1. First Change

First, a first change will be described. In a case where the change condition is satisfied, the biological information measurement system 1 performs the first change of changing a value included in the first information to a preset setting value. The biological information measurement system 1 performs the first change in a case where the first calculation value or the second calculation value is larger than the first threshold or in a case where the third calculation value is smaller than the second threshold smaller than the first threshold. In this case, the biological information measurement system 1 performs the first change in a case where at least one of change conditions including a first condition that the first calculation value or the second calculation value is larger than the first threshold and a second condition that the third calculation value is smaller than the second threshold smaller than the first threshold is satisfied.

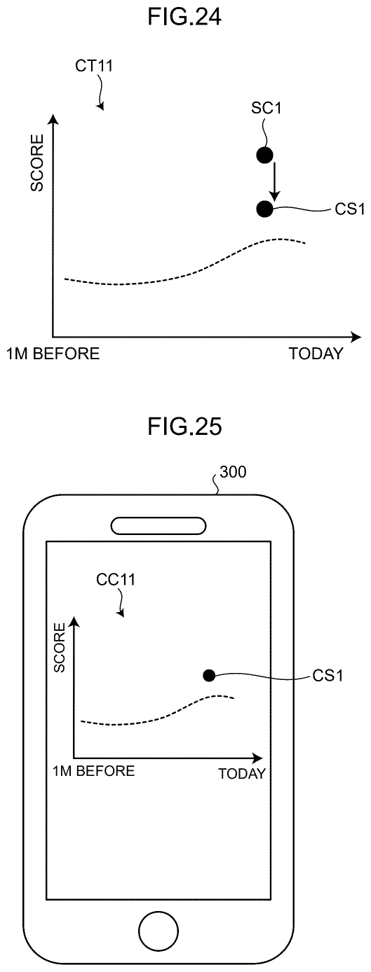

The biological information measurement system 1 performs the first change as illustrated in FIG. 24. FIG. 24 is a diagram illustrating the first change of information by the biological information measurement system. A content CT11 in FIG. 24 is information indicating a temporal change in the score based on the measurement of the fecal gas of the user.

A score SC1 in the content CT11 indicates a score before the change based on the measurement (also referred to as “target measurement”) at a corresponding date and time. The biological information measurement system 1 performs the first change in a case where any one of values obtained in the target measurement satisfies at least one of the change conditions. In a case where it is determined that a value obtained in the target measurement satisfies the change condition, the biological information measurement system 1 performs the first change.

In FIG. 24, the biological information measurement system 1 determines that the value obtained in the target measurement satisfies the change condition, and changes the score SC1 that is original data in the content CT11. For example, the biological information measurement system 1 changes the score SC1 to a changed score CS1 having a predetermined value. An arbitrary value may be set as the changed score CS1, which will be described below. In FIG. 24, by changing the score SC1 to the changed score CS1, the biological information measurement system 1 can set the score corresponding to the target measurement to a value close to an average indicated by a dotted line (such as a moving average indicating a temporal variation tendency).

The biological information measurement system 1 outputs information (a content CC11 in FIG. 25) including the changed score CS1 obtained by changing the score SC1 that is the original data. The biological information measurement system 1 outputs (transmits) the information including the changed score CS1 obtained by changing the score SC1 that is the original data to the display means 300 that is the user terminal used by the user (also referred to as a “user X”) who is a target in the target measurement.

The display means 300 that is used by the user X and has received the information including the changed score CS1 output by the biological information measurement system 1 displays the information as illustrated in FIG. 25. FIG. 25 is a diagram illustrating a display example of the information after the change by the biological information measurement system. As illustrated in FIG. 25, the display means 300 used by the user X displays the content CC11 including the changed score CS1 obtained by changing the score SC1 that is the original data. As described above, the display means 300 used by the user X does not display the score SC1 that is the original data estimated to be greatly affected by the measurement variations, but displays the changed score CS1 estimated to be more appropriate than the score SC1 by using such alternative information. In this manner, the biological information measurement system 1 may change information to be displayed to a predetermined value.

As described above, the biological information measurement system 1 can change information to be provided to the user by performing the first change, thereby suppressing the influence of the measurement variations. Therefore, the biological information measurement system 1 can appropriately execute processing based on the gas measurement.

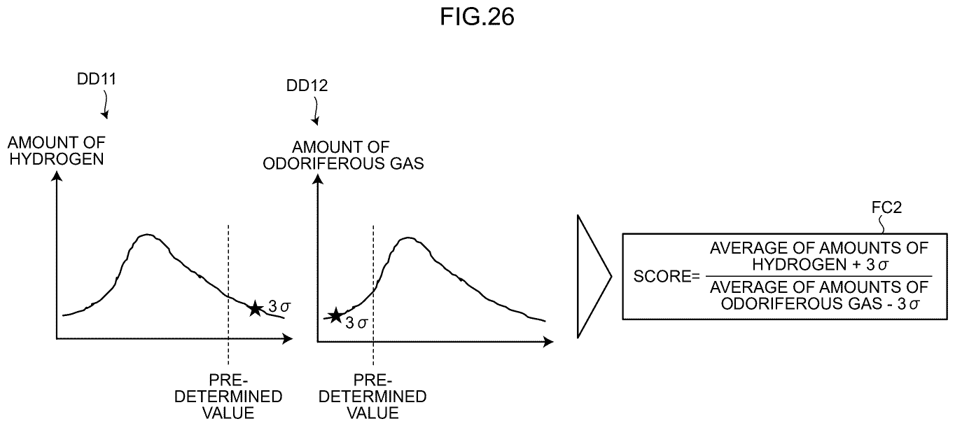

The information after the change, such as a value of the changed score CS1, may be set to arbitrary information. An example of this matter will be described with reference to FIG. 26. FIG. 26 is a diagram illustrating a score correction example of the biological information measurement system. In FIG. 26, the information after the change may be determined using hydrogen amount distribution information DD11 indicating a distribution of an amount of hydrogen emitted from a person and odoriferous gas amount distribution information DD12 indicating a distribution of the amount of the odoriferous gas emitted from a person.

For example, the biological information measurement system 1 may calculate a score when the first calculation value is a predetermined value and the second calculation value is a predetermined value. For example, the biological information measurement system 1 may calculate the score from a maximum amount/minimum amount (such as 3σ of the distribution) from a human.

For example, the biological information measurement system 1 may calculate the changed score CS1 by using a function FC2. The function FC2 may use, as the changed score CS1, a value obtained by dividing a value obtained by adding 38 to an average of the amounts of hydrogen calculated based on the hydrogen amount distribution information DD11 by a value obtained by subtracting 38 from an average of the amounts of the odoriferous gas calculated based on the odoriferous gas amount distribution information DD12.

### 1-11-2. Second Change

The change performed by the biological information measurement system 1 is not limited to the first change, and the biological information measurement system 1 may perform the change using the arbitrary information. For example, the biological information measurement system 1 may perform a second change of changing the information by using past information of a target user. In this case, the biological information measurement system 1 stores the first information output based on the past measurement in the storage unit 120. Specifically, the biological information measurement system 1 stores the history information including the score calculated based on the past measurement in the storage unit 120.

The biological information measurement system 1 performs the second change of changing the information based on the score calculated based on the past measurement stored in the storage unit 120. The biological information measurement system 1 performs the second change in a case where at least one of the change conditions is satisfied.

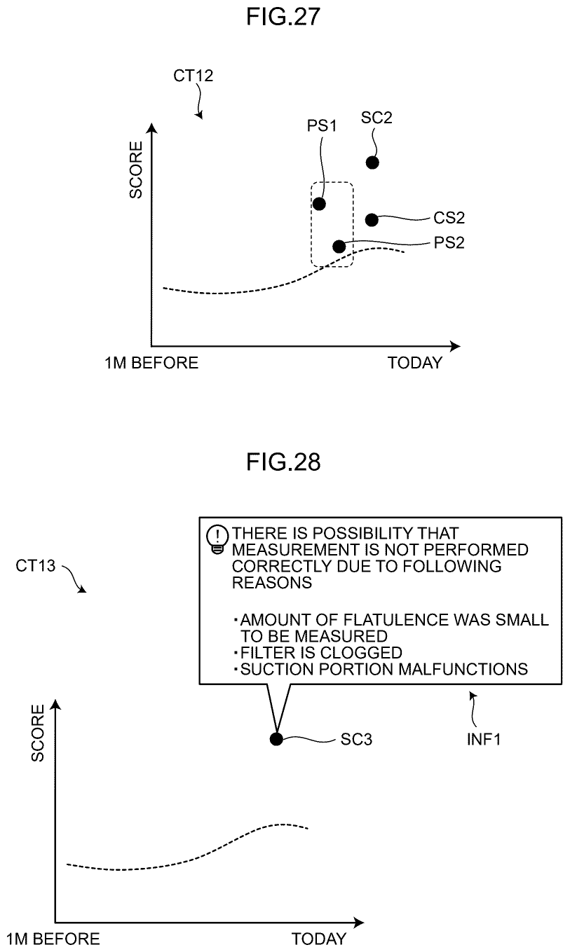

The biological information measurement system 1 performs the second change as illustrated in FIG. 27. FIG. 27 is a diagram illustrating the second change of the information by the biological information measurement system. A content CT12 in FIG. 27 is information indicating a temporal change in the score based on the measurement of the fecal gas of the user.

A score SC2 in the content CT12 indicates a score before the change based on the measurement (target measurement) at a corresponding date and time. The biological information measurement system 1 performs the second change in a case where any one of values obtained in the target measurement satisfies at least one of the change conditions. In a case where it is determined that a value obtained in the target measurement satisfies the change condition, the biological information measurement system 1 performs the second change.

In FIG. 27, the biological information measurement system 1 determines that the value obtained in the target measurement satisfies the change condition, and changes the score SC2 that is original data in the content CT12. For example, in a case where it is determined that the value obtained in the target measurement satisfies the change condition, the biological information measurement system 1 changes the value to a changed score CS2 having a value calculated using a plurality of scores corresponding to measurement performed before the target measurement. As the changed score CS2, an average value of the plurality of scores corresponding to the most recent plurality of times of measurement performed before the target measurement is used. In FIG. 27, the biological information measurement system 1 calculates the changed score CS2 by using the latest scores PS1 and PS2 before the target measurement.

As a result, the information change is performed based on the past tendency of the user (user X) who is the target in the target measurement, whereby the biological information measurement system 1 can change the information to information based on the past tendency of the user X. For example, it is assumed that the scores PS1 and PS2 do not satisfy the change condition and are scores as original data. For example, the biological information measurement system 1 outputs a score that is not changed (corrected) for data that has not been changed, such as the score PS1 or the score PS2.

The biological information measurement system 1 outputs information including the changed score CS2 obtained by changing the score SC2 that is the original data. The biological information measurement system 1 outputs (transmits) the information including the changed score CS2 obtained by changing the score SC2 that is the original data to the display means 300 that is the user terminal used by the user (user X) who is the target in the target measurement. Matters regarding the display of the information are similar to the content described in FIG. 25, and thus, a detailed description thereof will be omitted.

As described above, the biological information measurement system 1 can change information to be provided to the user by performing the second change, thereby suppressing the influence of the measurement variations. Therefore, the biological information measurement system 1 can appropriately execute processing based on the gas measurement.

In this manner, the biological information measurement system 1 corrects information to be displayed by referring to the past data. The biological information measurement system 1 displays an average value by referring to data of a plurality of times of measurement in the past. The biological information measurement system 1 displays an average value obtained by giving a greater weight to more recent data, by referring to the data of the plurality of times of measurement in the past. The biological information measurement system 1 is not limited to a case of using an average value of pieces of data of the most recent two times of measurement as described above, and may use arbitrary information. For example, the biological information measurement system 1 may use a weighted average of pieces of data of the most recent three or more times of measurement. In this case, the biological information measurement system 1 may use a weighted average obtained by giving a greater weight to more recent data among the pieces of data of the most recent three or more times of measurement. In addition, the biological information measurement system 1 does not need to use information changed in the past (changed score) when calculating the average value.

### 1-11-3. Third Change

The change performed by the biological information measurement system 1 is not limited to the first change and the second change, and the biological information measurement system 1 may perform the change using the arbitrary information. For example, the biological information measurement system 1 may perform a third change of changing the information by using notification information for the user. In this case, in a case where a predetermined condition is satisfied, the biological information measurement system 1 outputs the second information regarding the measurement accuracy. In a case where the predetermined condition is satisfied, the biological information measurement system 1 outputs the third information regarding the measurement error.

The biological information measurement system 1 performs the third change in a case where at least one of the change conditions is satisfied. The biological information measurement system 1 performs the third change as illustrated in FIG. 28. FIG. 28 is a diagram illustrating the third change of the information by the biological information measurement system. A content CT13 in FIG. 28 is information indicating a temporal change in the score based on the measurement of the fecal gas of the user.

A score SC3 in the content CT13 indicates a score before the change based on the measurement (target measurement) at a corresponding date and time. The biological information measurement system 1 performs the third change in a case where any one of values obtained in the target measurement satisfies at least one of the change conditions. In a case where it is determined that a value obtained in the target measurement satisfies the change condition, the biological information measurement system 1 performs the third change.

In FIG. 28, the biological information measurement system 1 determines that the value obtained in the target measurement satisfies the change condition, and adds information INF1 to the content CT13. For example, in a case where it is determined that the value obtained in the target measurement satisfies the change condition, the biological information measurement system 1 adds, to the content CT13, the second information indicating that attention is required for the value calculated by the target measurement. The biological information measurement system 1 adds the information INF1 including information indicating that the measurement accuracy may be poor to the content CT13. The biological information measurement system 1 adds the information INF1 including the third information indicating that there is a possibility that the measurement error has occurred to the content CT13.

The biological information measurement system 1 outputs the content CT13 to which the information INF1 including the second information regarding the measurement accuracy and the third information regarding the measurement error is added. The biological information measurement system 1 outputs (transmits) the content CT13 to which the information INF1 for notifying the user of a possibility that there is a problem in the measurement of the score SC3 of the target measurement is added to the display means 300 that is the user terminal used by the user (user X) who is the target in the target measurement. Matters regarding the display of the information are similar to the content described in FIG. 25, and thus, a detailed description thereof will be omitted.

As described above, the biological information measurement system 1 can change information to be provided to the user by changing the information by performing the third change, thereby suppressing the influence of the measurement variations. Therefore, the biological information measurement system 1 can appropriately execute processing based on the gas measurement.

The biological information measurement system 1 may also perform the third change in a case where a condition other than the above-described change condition is satisfied. For example, the biological information measurement system 1 may perform the third change of adding the information INF1 to the content CT13 in order to notify that there is a possibility that the measurement has not been correctly performed, like a case where the measurement itself is difficult.

The above-described embodiment and modified examples can be appropriately combined within a range not resulting in contradictions in processing.

Further effects and modified examples can be easily derived by those skilled in the art. Therefore, broader aspects of the present invention are not limited to the specific details and representative embodiments presented and described above. Accordingly, various changes may be made without departing from the spirit or scope of the general inventive concept as defined by the appended claims and equivalents thereof.

The above-described embodiment and modified examples may have the following configurations, but are not limited to the following configurations.

(1)

A biological information measurement system that measures biological information of a user in a toilet room based on fecal gas emitted into a bowl of a closet bowl installed in the toilet room, the biological information measurement system including:
a gas detection device that includes a first gas sensor that reacts with a hydrogen gas contained in a gas mixture, and a second gas sensor that reacts with an odoriferous gas containing a sulfur component and the hydrogen gas; and
a control device that controls the gas detection device, in which
the control device
calculates a second calculation value corresponding to the hydrogen gas of the second gas sensor based on a plurality of calculation values corresponding to the hydrogen gas contained in the gas mixture, and
calculates a third calculation value corresponding to the odoriferous gas based on a detection result of the second gas sensor and the second calculation value,
the biological information measurement system estimates a health condition of the user or information regarding the health condition based on the third calculation value, and
the plurality of calculation values include a first calculation value corresponding to the hydrogen gas obtained based on a detection result of the first gas sensor.
   (2)

The biological information measurement system according to (1), including:
a suction device that sucks the gas mixture in the bowl; and
sealing means that holds a gas sucked by the suction device in a sealed space, wherein
the second calculation value is a statistical value of the plurality of calculation values obtained by measuring the gas in the sealed space a plurality of times by using the first gas sensor.
   (3)

The biological information measurement system according to (2), in which
the sealing means includes a storage portion that stores the gas sucked by the suction device, and
the second calculation value is a statistical value of the plurality of calculation values obtained by measuring the gas in the storage portion a plurality of times.
   (4)

The biological information measurement system according to (2), in which
the sealing means has a flow path switchable to a closed flow path by opening/closing means, and the second calculation value is a statistical value of
the plurality of calculation values obtained by measuring a gas in the flow path a plurality of times.
   (5)

The biological information measurement system according to any one of (1) to (4), including
a third gas sensor that has higher sensitivity to the hydrogen gas and lower sensitivity to the odoriferous gas than the second gas sensor, wherein
the plurality of calculation values include the first calculation value and a fourth calculation value corresponding to the hydrogen gas obtained based on a detection result of the third gas sensor.
   (6)

The biological information measurement system according to (5), in which
the third gas sensor is a gas sensor that reacts with a methane gas, and
the control device
uses the third gas sensor as a sensor for detecting the methane gas in a case where it is determined that the methane gas is contained in the fecal gas of the user, and
uses the third gas sensor as a sensor for detecting the hydrogen gas in a case where it is determined that the methane gas is not contained in the fecal gas of the user.
   (7)

A biological information measurement system that measures biological information of a user in a toilet room based on fecal gas emitted into a bowl of a closet bowl installed in the toilet room, the biological information measurement system including:
a gas detection device that includes a first gas sensor that reacts with a hydrogen gas contained in a gas mixture, and a second gas sensor that reacts with an odoriferous gas containing a sulfur component and the hydrogen gas; and
a control device that controls the gas detection device, in which
the control device
calculates a second calculation value corresponding to the hydrogen gas of the second gas sensor based on a plurality of calculation values corresponding to the hydrogen gas contained in the gas mixture, and
calculates a third calculation value corresponding to the odoriferous gas based on a detection result of the second gas sensor and the second calculation value,
the biological information measurement system estimates a health condition of the user or information regarding the health condition by using a score calculated based on the third calculation value, and
the score is calculated based on a ratio between a value based on a first calculation value corresponding to the hydrogen gas obtained based on a detection result of the first gas sensor corresponding to detection at a common timing in defecation of the user, and a value based on the third calculation value.
   (8)

A toilet seat device that measures biological information of a user in a toilet room based on fecal gas emitted into a bowl of a closet bowl installed in the toilet room, the toilet seat device including:
a gas detection device that includes a first gas sensor that reacts with a hydrogen gas contained in a gas mixture, and a second gas sensor that reacts with an odoriferous gas containing a sulfur component and the hydrogen gas; and
a control device that controls the gas detection device, in which
the control device
calculates a second calculation value corresponding to the hydrogen gas of the second gas sensor based on a plurality of calculation values corresponding to the hydrogen gas contained in the gas mixture, and
calculates a third calculation value corresponding to the odoriferous gas based on a detection result of the second gas sensor and the second calculation value,
the toilet seat device estimates a health condition of the user or information regarding the health condition based on the third calculation value, and
the plurality of calculation values include a first calculation value corresponding to the hydrogen gas obtained based on a detection result of the first gas sensor.
   (9)

A toilet seat device that measures biological information of a user in a toilet room based on fecal gas emitted into a bowl of a closet bowl installed in the toilet room, the toilet seat device including:
a gas detection device that includes a first gas sensor that reacts with a hydrogen gas contained in a gas mixture, and a second gas sensor that reacts with an odoriferous gas containing a sulfur component and the hydrogen gas; and
a control device that controls the gas detection device, in which
the control device
calculates a second calculation value corresponding to the hydrogen gas of the second gas sensor based on a plurality of calculation values corresponding to the hydrogen gas contained in the gas mixture, and
calculates a third calculation value corresponding to the odoriferous gas based on a detection result of the second gas sensor and the second calculation value,
the toilet seat device estimates a health condition of the user or information regarding the health condition by using a score calculated based on the third calculation value, and
the score is calculated based on a ratio between a value based on a first calculation value corresponding to the hydrogen gas obtained based on a detection result of the first gas sensor corresponding to detection at a common timing in defecation of the user, and a value based on the third calculation value.

### REFERENCE SIGNS LIST

1 BIOLOGICAL INFORMATION MEASUREMENT SYSTEM
2 TOILET SEAT DEVICE
3 MAIN BODY PORTION
4 MEASUREMENT DEVICE
5 TOILET SEAT
6 CLEANING NOZZLE
7 TOILET BOWL
8 BOWL PORTION
9 TOILET LID
10 SUCTION DEVICE
20 GAS DETECTION DEVICE
40 GAS SENSOR
100 CONTROL DEVICE
110 COMMUNICATION UNIT
120 STORAGE UNIT
130 CONTROL UNIT
131 ACQUISITION UNIT
132 PROCESSING UNIT
133 OUTPUT UNIT
200 ESTIMATION MEANS
R TOILET ROOM

What is claim is:

1. A biological information measurement system that measures biological information of a user in a toilet room based on intestinal gas emitted into a bowl of a closet bowl installed in the toilet room, the biological information measurement system comprising:
a gas detection device that includes a first gas sensor that reacts with a hydrogen gas contained in a gas mixture, and a second gas sensor that reacts with an odoriferous gas containing a sulfur component and the hydrogen gas; and
a control device that controls the gas detection device, wherein
the control device
calculates a second calculation value corresponding to the hydrogen gas of the second gas sensor based on a plurality of calculation values corresponding to the hydrogen gas contained in the gas mixture, and
calculates a third calculation value corresponding to the odoriferous gas based on a detection result of the second gas sensor and the second calculation value,
the biological information measurement system estimates a health condition of the user or information regarding the health condition based on the third calculation value, and
the plurality of calculation values include a first calculation value corresponding to the hydrogen gas obtained based on a detection result of the first gas sensor.

2. The biological information measurement system according to claim 1, comprising:
a suction device that sucks the gas mixture in the bowl; and
sealing means that holds a gas sucked by the suction device in a sealed space, wherein
the second calculation value is a statistical value of the plurality of calculation values obtained by measuring the gas in the sealed space a plurality of times by using the first gas sensor.

3. The biological information measurement system according to claim 2, wherein
the sealing means includes a storage portion that stores the gas sucked by the suction device, and
the second calculation value is a statistical value of the plurality of calculation values obtained by measuring the gas in the storage portion a plurality of times.

4. The biological information measurement system according to claim 2, wherein
the sealing means has a flow path switchable to a closed flow path by opening/closing means, and
the second calculation value is a statistical value of the plurality of calculation values obtained by measuring a gas in the flow path a plurality of times.

5. The biological information measurement system according to claim 1, comprising
a third gas sensor that has higher sensitivity to the hydrogen gas and lower sensitivity to the odoriferous gas than the second gas sensor, wherein
the plurality of calculation values include the first calculation value and a fourth calculation value corresponding to the hydrogen gas obtained based on a detection result of the third gas sensor.

6. The biological information measurement system according to claim 5, wherein
the third gas sensor is a gas sensor that reacts with a methane gas, and
the control device
uses the third gas sensor as a sensor for detecting the methane gas in a case where it is determined that the methane gas is contained in the intestinal gas of the user, and
uses the third gas sensor as a sensor for detecting the hydrogen gas in a case where it is determined that the methane gas is not contained in the intestinal gas of the user.

7. A biological information measurement system that measures biological information of a user in a toilet room based on intestinal gas emitted into a bowl of a closet bowl installed in the toilet room, the biological information measurement system comprising:
a gas detection device that includes a first gas sensor that reacts with a hydrogen gas contained in a gas mixture, and a second gas sensor that reacts with an odoriferous gas containing a sulfur component and the hydrogen gas; and
a control device that controls the gas detection device, wherein
the control device
calculates a second calculation value corresponding to the hydrogen gas of the second gas sensor based on a plurality of calculation values corresponding to the hydrogen gas contained in the gas mixture, and
calculates a third calculation value corresponding to the odoriferous gas based on a detection result of the second gas sensor and the second calculation value,
the biological information measurement system estimates a health condition of the user or information regarding the health condition by using a score calculated based on the third calculation value, and
the score is calculated based on a ratio between a value based on a first calculation value corresponding to the hydrogen gas obtained based on a detection result of the first gas sensor corresponding to detection at a common timing in defecation of the user, and a value based on the third calculation value.

8. (canceled)

9. A toilet seat device that measures biological information of a user in a toilet room based on intestinal gas emitted into a bowl of a closet bowl installed in the toilet room, the toilet seat device comprising:
a gas detection device that includes a first gas sensor that reacts with a hydrogen gas contained in a gas mixture, and a second gas sensor that reacts with an odoriferous gas containing a sulfur component and the hydrogen gas; and
a control device that controls the gas detection device, wherein
the control device
calculates a second calculation value corresponding to the hydrogen gas of the second gas sensor based on a plurality of calculation values corresponding to the hydrogen gas contained in the gas mixture, and
calculates a third calculation value corresponding to the odoriferous gas based on a detection result of the second gas sensor and the second calculation value,
the toilet seat device estimates a health condition of the user or information regarding the health condition by using a score calculated based on the third calculation value, and
the score is calculated based on a ratio between a value based on a first calculation value corresponding to the hydrogen gas obtained based on a detection result of the first gas sensor corresponding to detection at a common timing in defecation of the user, and a value based on the third calculation value.
